(19) United States
(12) Patent Application Publication
(10) Pub. No.: US 2019/0275175 A1
(43) Pub. Date: Sep. 12, 2019

(54) Carbon Nanotubes for Imaging and Drug Delivery

(71) Applicant: The Board of Trustees of the Leland Stanford Junior University, Stanford, CA (US)

(72) Inventors: Bryan R. Smith, Palo Alto, CA (US); Eliver Ghosn, Palo Alto, CA (US)

(21) Appl. No.: 16/353,743

(22) Filed: Mar. 14, 2019

Related U.S. Application Data
(62) Division of application No. 14/020,794, filed on Sep. 7, 2013, now abandoned.
(60) Provisional application No. 61/698,242, filed on Sep. 7, 2012.

(51) Int. Cl. A61K 49/00; A61K 47/69; B82Y 5/00; A61K 47/60; A61K 47/64

(52) U.S. Cl. CPC A61K 49/0056; A61K 47/6949; A61K 47/64; A61K 47/60; B82Y 5/00

(57) ABSTRACT

The invention provides compositions and methods for visualizing particular tissues and delivering one or more therapeutics to that tissue using single-walled carbon nanotubes (SWNTs), which are taken up and delivered to target tissues by specific monocytes in the body. The delivery of SWNT to target tissues allows the visualization of the affected tissue for diagnostics and therapy in diseases where the specific monocyte is implicated in the disease pathogenesis. These nanotubes can be conjugated to a peptide, such as RGD, which helps direct the SWNT-containing monocytes to the vascular endothelium.

### CROSS-REFERENCE TO RELATED APPLICATIONS

The present application claims priority to U.S. patent application Ser. No. 14/020,794, filed on Sep. 7, 2013, which claims the benefit U.S. Provisional Patent Application Ser. No. 61/698,242, filed Sep. 7, 2012, which applications are incorporated herein by reference in their entireties.

### STATEMENT OF GOVERNMENT SUPPORT

This invention was made with Government support under NIH CA151459, CA119367 and CA160764 awarded by the National Institutes of Health. The Government has certain rights in the invention.

### TECHNICAL FIELD OF THE INVENTION

The present invention relates to the field of imaging and drug delivery. In particular, it relates to the use of carbon nanotubes for visualizing particular tissues and delivering therapeutic treatment to that tissue.

### BACKGROUND

The effort to determine the existence and location of a disease in a human subject has a long history. Recently, procedures have advanced for more precisely locating specific tissue in a body. Human subjects are screened regularly for a variety of particular tumors such as colon, breast, prostate, etc. The screening techniques can be visual, such as for colon tumor, where an endoscope is inserted into the human subject and the surface of the colon is studied via a camera on the tip of the endoscope. For breast tumor, palpation, mammography and ultrasonography are regularly used. For prostate tumor, palpation, and antigen detection testing for PSA (prostate specific antigen), a practice that has become controversial (Andriole et al., 2009), have been used. In some of these procedures screening can lead to false positives as well as false negatives, depending on the test used. It can also lead to unnecessary invasive tests on a human subject when a false positive exists. When there is a family history of a disease, or manifestation of a particular symptom related to a particular type of tumor, more intense and invasive tests have been deemed appropriate. However, it is often very difficult to specifically locate a tumor and such efforts at localization can be very invasive. Often a biopsy is required to confirm that what “looks” like a malignant tumor actually is one.

Coronary artery atherosclerosis can be fatal in both men and women due to unstable plaque growth and sudden atherothrombotic events due to thrombosis caused by unstable plaques. Plaques, made of fat, cholesterol, calcium and other substances from the blood, build up in the blood vessels, resulting in narrowing, and ultimately blocking of these vessels that are needed to oxygenate and remove CO2 from the body's tissue. More importantly, some of such plaques can be unstable, meaning that they are vulnerable to rupture at any moment causing, for example, a heart attack.

Several invasive tests exist for detecting atherosclerosis, including CT scanning with contrast agent, MRI, and angiography. Lindner (2010) reported about the use of molecular imaging of myocardial and vascular disorders to detect VCAM-1, a vascular adhesion molecule that appears in the vasculature at the beginning of inflammation, already in the early stages of atherosclerosis. The procedure detects microbubbles that adhere to the VCAM-1 molecules and are visualized using contrast-enhanced ultrasound. While this is a positive move toward early diagnosis of atherosclerosis, testing has so far only been in animals, and the process may not work in humans.

Carbon nanotubes have a wide range of commercial applications including uses in electronic devices, electromechanical actuators, electrochemical sensors, drug delivery systems and more. While carbon nanotubes hold great potential for those various uses, they are often compared to asbestos, and their safety profile for human use and biocompatibility with human tissues can still be a matter of concern for some (Endo et al., 2008).

Since that time, studies have found that the toxicity of carbon nanotubes depends on the shape, size and surface of the structures (Schipper et al., 2008). For example, it was shown that single-walled carbon nanotubes (SWNTs) functionalized by PEGylated phospholipids are non-toxic over a period of at least 144 days (Robinson et al., 2010).

While the Robinson work shows the safety of using SWNTs in imaging and photothermal tumor treatment, it does not address the issue of locating a tumor or other tissue in order to diagnose the presence or absence of damaged tissue in a particular location. Nor does Robinson teach monitoring or treatment of such tissue, as the Robinson techniques relied on knowing where a mouse tumor existed in order to treat it.

It would be highly desirable to have compositions and methods available to visualize particular tissues and to deliver therapeutic treatment to a mammalian subject, including a human subject.

### SUMMARY OF THE INVENTION

The present invention translates the study of nanoparticles such as single-walled carbon nanotubes (SWNTs) into clinical use by employing them for molecular imaging. Successful delivery of the carbon nanotubes is critical for their effective use in humans. For example, in order to treat a tumor, the nanoparticles must reach and interact with the tumor. This invention, including its variations, utilizes the ability of SWNTs to precisely reach and enter tumors, and to provide chemotherapy to those tumors.

In one aspect, the present invention provides a method, using single-walled carbon nanotubes, to localize a tissue of interest in a living mammalian body. In one embodiment, the tissue of interest is a tumor having a vasculature system. In a related embodiment, the tissue of interest is a malignant tumor. In another embodiment, the tissue of interest is atherosclerotic tissue. In another embodiment, SWNTs are derivatized with peptides and delivered to a living mammalian subject including a human subject, to localize the subject's vasculature.

In an additional embodiment, SWNTs are taken up by a distinct set of monocytes in the mammalian subject's vasculature. In a further embodiment, the monocytes are Ly-6C^{hi} monocytes into which the derivatized SWNTs very specifically enter. In yet another embodiment, the SWNTs are derivatized with small peptides before they are delivered to the human subject. In one embodiment, the derivatized SWNTs are in the Ly-6C^{hi} monocytes and are visualized using a variety of visualization techniques. In an embodiment, the derivatized SWNTs in the Ly-6C^{hi} monocytes provide the existence and location of the tissue, such as a tumor, by the location of the SWNTs. In another embodiment, the derivatized SWNTs in the Ly-6C^{hi} monocytes provide the existence and location of atherosclerotic tissue in a human subject, by the location of the SWNTs. In another embodiment, the existence and location of a tumor in a human subject having CD14+ monocytes (human counterpart for murine Ly6-C^{hi} monocytes) is determined. In another embodiment, the derivatized SWNTs in the Ly6-C^{hi} monocytes carry one or more therapeutic compositions to the tumor. In a further aspect, the existence and location of atherosclerotic tissue in a human subject having CD14⁺ monocytes is determined. In another embodiment, the derivatized SWNTs in the Ly-6C^{hi} monocytes or CD14⁺ monocytes carry therapeutic compositions to the atherosclerotic tissue.

In another embodiment, CD14⁺ monocytes and their progenitors, including macrophages, neutrophils and dendritic cells, are selectively differentiated via agents that are attached to the nanotubes and which facilitate the differentiation of CD14⁺ monocytes such as cytokines, including interleukins and interferons.

In a further embodiment, CD14⁺ monocytes are selectively destroyed via laser light irradiation of the single-walled carbon nanotubes that they carry.

### BRIEF DESCRIPTION OF THE FIGURES

FIG. 1 shows intravital microscopy on single-walled carbon nanotubes (SWNTs) within a mouse body; in this embodiment, an IV-100 intravital microscopy system (FIG. 1a/b) is pictured (FIG. 1a). Also shown are a mouse being imaged by intravital microscopy (FIG. 1b) by fixing the dorsal skinfold chamber (FIG. 1c) onto a stage.

FIG. 2 shows how SWNTs look without attached peptides (FIG. 2a) and with attached peptides (FIG. 2b). FIG. 2c shows an electron micrograph of a SWNT with dimensions similar to the ones used in embodiments herein.

FIG. 3 shows an EGFP-transduced tumor sample taken from a mouse several hours after injection of RGD-SWNTs. The images of the tumor (green), the blood vessels (circulating red dye) and the SWNTs (gray) have been merged to show the location of the SWNTs. Several monocytes carrying the RGD-SWNTs can be seen moving through the blood vessels (arrows designate several of them).

FIG. 4 compares the amount of uptake of plain and peptide-conjugated SWNTs into the mouse's circulating cells, calculated by direct count of the respective SWNTs, clearly demonstrating a difference in the kinetics of uptake into monocytes depending on the presence of a peptide.

FIG. 5 presents a different view of the kinetics of uptake by circulating cells of peptide-conjugated SWNTs versus plain SWNTs, as a function of the concentration of SWNTS performed in cell culture.

FIG. 6 shows a schematic of a mouse injected with SWNTs (FIG. 6a), the blood and organs from various regions having been harvested after the injection and used in studies represented in part in FIGS. 7 and 8. Arrows point to (nanotube-laden) monocytes moving through the mouse vasculature.

FIGS. 7a-d shows fluorescence activated cell sorting (FACS) results using twelve specific dyes to detect SWNTs present in blood monocyte cells. FIG. 7a shows blood cells after PBS injection (negative control). FIG. 7b-d shows blood cells 2 h after intravenous injection of SWNT. Ly-6C^{hi} monocytes are the only subset that internalized SWNTs in vivo. Blood monocytes represent 16% of total white blood cells after excluding neutrophils. The plots on the right show monocytes without SWNTs (PBS negative control). FIG. 7b shows that neutrophils do not uptake SWNTs 2 h after intravenous injection of SWNT. FIG. 7c shows that SWNTs were taken up in about 100% of the Ly-6C^{hi} monocytes. FIG. 7d shows that other myeloid cells such as Ly-6C^{lo} monocytes and dendritic cells do not internalize SWNTs.

FIG. 8 shows FACS results using twelve specific dyes to detect SWNTs present in monocyte cells from the spleen of the mouse body. Over 90% of the Ly-6C^{hi} monocytes in the spleen take up SWNTs (93.4% in the specimen are shown in FIG. 8a). Controls show no signal in Ly-6C^{hi} monocytes when the mouse is injected with vehicle (PBS) containing no SWNTs (FIG. 8b).

FIG. 9 illustrates that monocytes, but not other immune cells, are specific targets of SWNTs. Here, to extract a hierarchy from high-dimensional flow cytometry data in an unsupervised manner, a computational approach was used, “spanning-tree progression analysis of density normalized events (SPADE)”. SPADE allowed visualization and representation of SWNT selectivity to Ly-6C^{hi} monocytes in spleen 2 hours after SWNT injection. Panel A: Red dots represent immune cells that express Ly-6C, i.e. inflammatory Ly-6C^{hi} monocytes. All other immune cells (neutrophils, B cells, T cells) do not express Ly-6C. Panel B: Red dots represent immune cells that internalized SWNT. Ly-6C^{hi} monocytes exclusively internalized SWNTs. Other immune and phagocytic (neutrophils) cells did not internalize SWNTs.

FIG. 10 shows the kinetics of SWNT internalization by immune cells in blood and spleen, where monocytes were found to selectively pick up SWNTs. The top panels show total live cells from blood at 2 h, 12 h, and 24 h after i.v. injection of SWNTs (PBS injection was used as control). At the earliest time of 2 hours following the injection of SWNTs, Ly-6C^{hi} (X axis) monocytes had already picked up SWNTs (Y axis). At 12 h post injection, virtually all circulating Ly-6C^{hi} monocytes had internalized SWNTs. At 24 h post injection, SWNTs were no longer detectable in blood. Red dotted lines represent the baseline fluorescence levels for SWNT channel in the control PBS-injected mice. Bottom panels show total live cells from spleen at the corresponding time-points shown above. Similarly to the Ly-6C^{hi} monocytes in blood, spleen Ly-6C^{hi} monocytes internalized SWNTs as fast as 2 hours post injection. The uptake peaked at 12 hours post injection and was barely detectable at 24 hours after SWNT injection.

FIG. 11 shows scatter plots of RNA-sequencing data (full transcriptome) for FACS-sorted, purified Ly-6C^{hi} monocytes at 6 hours after SWNT internalization in vivo. Data were compared to Ly-6C^{hi} monocytes that were purified from spleen, obtained from control PBS-injected mice. Names of the genes for some of the biggest differences between control and experimental groups are included on the plots. Both SWNT+ and SWNT− monocytes showed similar gene expression (transcriptome) profile at 6 hours after i.v. injection of SWNT or PBS, indicating a lack of activation by the SWNTs and suggesting that SWNT uptake was not detrimental to the cells.

FIG. 12 shows a merged view of tumor, blood vessels and SWNTs being taken into the tumor. The SWNTs in cells appear as small circular-to-elliptical structures as indicated by the arrow. As can be seen, most of the SWNT-monocytes are located along the endothelium of the blood vessels.

FIG. 13 shows monocytes that have been transported into the tumor interstitium (see arrows).

FIG. 14 shows the peptide dependence of tumor targeting. In comparison to RAD-SWNTs, the RGD peptide clearly caused a marked increase (p<0.0001) in targeting of SWNT-loaded cells to the tumor.

FIG. 15 shows the result of FACS of cells in the tumor mass at various time-points after intravenous injection of SWNTs. About 10%-20% of total cells in the tumor mass represent myeloid (CD11b+) cells. The remaining 80%-90% are tumor cells.

FIG. 16 shows further FACS analysis on the myeloid cells gated from FIG. 15. FIGS. 16a-c show that about 1% of total myeloid cells in tumor mass represent neutrophils (upper right corner of FACS plots, CD11b^{hi}, Gr-1^{hi}).

FIG. 17 shows that the remaining 98%-99% of myeloid cells in the tumor (excluding 1% of neutrophils shown in FIG. 16) represent Ly-6C^{hi} monocytes. FACS plots show that total Ly-6C^{hi} monocytes that infiltrated the tumor mass decrease their surface expression of Ly-6C to become tumor macrophages.

FIG. 18 shows the surface expression level of MHC-II on Ly-6Chi monocytes that are infiltrated into the tumor mass several hours after SWNT injection. After 24 h (lower right plot) Ly-6Chi monocytes in the tumor represent two subsets based on MHC-II expression (MHC-II and MHC-II⁺).

FIG. 19 shows the Ly-6C^{hi} monocytes that enter the tumor mass up to 12 h after SWNTs injection have internalized SNWTs (FACS plots for 12 h). However, Ly-6C^{hi} monocytes that infiltrated the tumors 24 h after injection (the monocytes that express highest levels of Ly-6C on FACS plots) are negative for SWNTs as these SWNTs are no longer detected in blood at this time-point.

FIG. 20 illustrates that Ly-6C^{hi} monocytes selectively picked up SWNTs and infiltrated the tumor mass in the cancer murine models. Tumors were processed into single cell suspensions and analyzed by Hi-Dimension FACS using 14 parameters simultaneously, namely Ly-6C, I-A/I-E, CD5, CD19, CD11b, Gr-1, CD45, SWNT-Cy55, Propidium Iodide to discriminate live from dead cells, CD80/CD86, Forward and Side Scatter to determine size and granularity, respectively, NK1.1, CD49b, F4/80. Plots show total live tumor cells 2 h, 12 h, and 24 h after i.v. injection of SWNTs (PBS injection was used as control). Top panels show that about 10-20% of total tumor cell fraction represented myeloid cells (CD11b+). Center panels show that about 2-15% of the tumor myeloid cells represented neutrophils (Gr-1^{hi}). Bottom panels show that Ly-6C^{hi} monocytes internalized SWNTs in a time-dependent manner. Unlike the Ly-6C^{hi} monocytes in blood and spleen, the tumor monocytes continued to accumulate SWNTs even at 24 hours after i.v. injection of SWNTs.

FIG. 21 illustrates that SWNTs were selectively internalized by foamy cells and Ly-6C^{hi} monocytes in atheroma plaques providing a good model for imaging of atheroma plaques. Plots represent the Hi-D FACS analysis of single cell suspension of total carotid artery following enzymatic digestion. Macrophage-rich atherosclerotic lesions were created as described by Kosuge et al., 2012. In brief, 8 wk-old male FVB mice were fed a high-fat diet containing 40% kcal fat, 1.25% (by weight) cholesterol, and 0.5% (by weight) sodium cholate. After a month, diabetes was induced by 5 daily intraperitoneal injections of streptozotocin (STZ; 40 mg/kg). Two weeks after the initiation of STZ injection, the left common carotid artery was ligated below the bifurcation. Sham operation was performed by passing the suture under the left carotid artery without constricting the artery. Two weeks after ligation, the left, diseased artery developed atheroma plaques and was harvested for Hi-D FACS analysis. The right, healthy artery that had not been ligated was also harvested and used as control. Top panels show all of the immune cells present in the right, healthy artery. The center panels show that there was a massive increase in foamy cells and other lymphocytes in the diseased, left artery. It can also be observed that neutrophils (Gr-1^{hi}, X axis) did not internalize SWNTs (Y axis). The bottom panels show that only foamy cells (which are macrophages derived from Ly-6C^{hi} monocytes) and Ly-6C^{hi} monocytes themselves internalized SWNTs 6 hours after i.v. injection. The bottom left panels show that T and B cells did not internalize SWNTs.

FIG. 22 illustrates that SWNTs can be used to detect atheroma plaques by imaging. 48 hours after in vivo injection of SWNTs, the left (diseased, ligated) artery and the right (control, not ligated) artery were harvested and analyzed in a fluorescent microscopy (MAESTRO instrument). Shown are the diseased and control arteries harvested from 3 mice 48 hours after injection of SWNTs in vivo. SWNTs migrated more efficiently to the diseased arteries, when compared to the control (not ligated) artery and remained in situ for 48 hours after i.v. injection of the SWNTs.

In FIG. 23, photoacoustic imaging of diseased versus healthy arteries in mice is illustrated as an alternative detection methodology for SWNTs using a model of vulnerable plaque which is a precursor to heart attacks. Panel A shows a schematic of a photoacoustic probe interrogating a diseased artery. In humans, probes can be incorporated into already-employed IVUS (intravascular ultrasound) probes and used to functionally identify vulnerable plaques after injection of the SWNTs. Panel B. Photoacoustic imaging, quantified here by n=3-4 mice per bar shown, showed very high signals in the arteries of mice at the site of disease, but significantly less signal in other sites of the same mouse and at the same site in normal mice (p<0.01 or lower for the 6 hour time-point vs. each of the other conditions). This was observed due to uptake of SWNTs into the Ly-6C^{hi} monocytes and into foamy macrophages, which are the cells into which Ly-6C^{hi} monocytes differentiate. Panel C. A three-dimensional visualization of the photoacoustic signal showed very strong signals in the diseased mouse artery at 6 hours post-injection of the SWNTs. The arrow designates the diseased artery loaded with SWNTs due to uptake by foamy macrophages and monocytes.

In FIG. 24, SWNTs were characterized in various ways. Panel A shows the zeta-potential (surface charge) of single-walled carbon nanotubes conjugated to Cy5.5. The charge distribution is shown alongside another common nanoparticle, quantum dots, showing very similar zeta-potential distributions. The peak of the distribution indicates that the SWNTs were approximately neutral. Panel B shows the absorption spectrum of SWNT-Cy5.5 (10 μl) in 200 μl PBS. Using these measurements, including subtraction of the PBS spectrum, it is shown that 10-300 Cy5.5 dyes can be conjugated per SWNT. In typical experiments, 15 dyes/SWNT were used. Panel C shows a TEM image of SWNTs dried onto a copper grid.

### DEFINITIONS

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The terms “tumor” and “tumors” refer to one or more lesions made by cells that have undergone abnormal growth. The tumor can be benign, pre-malignant or malignant. The tumor can also be vascularized or not. In embodiments of the present invention, the tumor can be malignant, pre-malignant or benign, as long as it is linked to the vasculature of the subject, presumably by undergoing angiogenesis. Malignant tumor and cancer are considered interchangeable terms in this application.

The term “mammalian subject” refers to a member of a species of mammalian origin, including but not limited to a human, mouse, rat, cat, goat, sheep, horse, hamster, ferret, pig, dog, guinea pig, rabbit or primate, adult or not yet adult.

The term “therapeutic”, as used herein, refers to a small molecule, nucleic acid, protein, peptide or other substance that provides a therapeutic effect, i.e. accomplishes one or more of the following: a) reduces the severity of a tumor or of atherosclerosis; b) limits the development of symptoms characteristic of the tumor or of the atherosclerosis treated; c) limits the worsening of symptoms characteristic of the tumor or of the atherosclerosis treated; d) limits the recurrence of the tumor or of the atherosclerosis treated and d) limits recurrence of symptoms of the tumor or of the atherosclerosis treated in mammalian subjects who were previously symptomatic for the tumor or atherosclerosis treated. The “therapeutic” is capable of damaging at least a portion of the cells within the tumorous or atherosclerotic tissue and/or driving those cells towards apoptosis, i.e. programmed death.

Monocytes are precursors for macrophages and dendritic cells, and, therefore, reference to cells as monocytes can include cells that have become macrophages; likewise, reference to cells as macrophages can include cells that are monocytes.

CD14⁺ monocytes, which are part of human peripheral blood, play an important role in innate as well as adaptive immunity through their ability to recognize pathogens, facilitate phagocytosis, and produce a wide array of immunomodulating agents, particularly cytokines such as IL-1β, IL-6 and IL-10 (Tiemessen et al., 2007).

RGD peptides, as understood herein, are peptides that contain the RGD tripeptide. The RGD tripeptide consists of L-arginine (standard amino acid abbreviation: Arg, R), glycine (Gly, G) and L-aspartic acid (Asp, D) and represents an essential attachment site for cell adhesion via integrin receptors, particularly via αᵥβ₃, which is capable of binding to a large variety of peptides and proteins that contain the RGD sequence.

RAD peptides, as understood herein, are peptides that contain the RAD tripeptide. The RAD tripeptide consists of L-arginine (Arg, R), alanine (Ala, A) and L-aspartic acid (Asp, D), in which one amino acid is different from RGD by the exchange of glycine for alanine. RAD peptides serve as a control for RGD peptides.

### DETAILED DESCRIPTION

Embodiments of the present invention describe the use of single-walled carbon nanotubes (SWNTs) for detecting as well as delivering treatment to tumorous or atherosclerotic tissue; the nanotubes can be functionalized with peptides such as RGD or peptides that contain the RGD-sequence. In the various embodiments of the present invention the SWNTs are selectively taken up by one particular group of monocytes that is present in the body, namely the Ly-6C^{hi} monocytes in mice and CD14⁺ monocytes in human subjects. The SWNTS of the invention specifically enter Ly-6C^{hi} monocytes and CD14⁺ monocytes, respectively, so that these monocytes pick up the SWNTs, and act in a different manner than other monocytes in the blood vessels.

In a further step, these SWNTs-carrying monocytes move then from the blood flow towards the blood vessel inner surface (the endothelium), ultimately interacting with this surface, moving along the surface and into a tumor, atherosclerotic tissue, and possibly other diseased tissue. This allows specific visualization and/or treatment of the diseased tissue where the monocytes and ultimately macrophages carrying the SWNTs gather. When the SWNTs, prior to being picked up by Ly-6C^{hi} monocytes or CD14+ monocytes, have been functionalized with a peptide specific for the vasculature in the targeted tissue, such as the RGD-peptide used in various embodiments herein, the above described process of moving into diseased tissue, such as tumorous or atherosclerotic tissue, is notably accelerated.

Monocytes are circulating blood cells that constitute approximately 10% of peripheral leukocytes (white blood cells) in humans (Yona et al., 2009). One of the subsets of monocytes are Ly-6C^{hi} monocytes. Monocytes develop in the bone marrow, and upon infection, a large number of Ly-6C^{hi} monocytes exit the bone marrow into the peripheral circulation. In fact, it appears that the total number of Ly-6C^{hi} monocytes increases upon infection. They naturally migrate to sites of inflammation, where the Ly-6C^{hi} monocytes can develop into macrophages and dendritic cells. It has also been found that in the absence of inflammation, the number of Ly-6C^{hi} monocytes in the peripheral blood decreases significantly. Thus, Ly-6C^{hi} monocytes naturally move toward inflamed tissue. In some embodiments of the present invention, this innate homing has been built upon to provide location information for tumors and atherosclerotic tissue, and to provide therapies for such tissue.

Single-Walled Carbon Nanotubes are Taken Up into Circulating Ly-6C^{hi} Monocytes and CD14+ Monocytes

Single-walled carbon nanotubes are used in the present invention for a number of purposes, several of which rely on the ability of certain SWNTs to travel into tumors, atherosclerotic tissue and other diseased tissue. When plain, i.e. non-conjugated SWNTs are delivered to the blood stream, the SWNTs are rapidly taken up by circulating Ly-6C^{hi} monocytes in mice and CD14⁺ monocytes in human subjects, respectively. When specific small peptides, such as the RGD peptide, are conjugated to the SWNTs, the resulting peptide-SWNTs are then not only taken up by monocytes such as Ly-6C^{hi} monocytes in mice and CD14⁺ monocytes in human subjects, but the peptide-SWNT-monocytes (i.e., the peptide-SWNT-Ly-6C^{hi} and peptide-SWNT-CD14⁺ monocytes) can also be directed to specific tissues.

The peptide-SWNT-Ly-6C^{hi} monocyte conjugates, for example, move in the blood stream, traveling around in the blood flow as do other blood cells. When they near the blood vessel endothelium, the peptides are attracted to a protein in the endothelium, and therefore enter into the endothelial tissue. The peptide-SWNT-Ly-6C^{hi} monocytes have been observed to travel along the blood vessel wall, and into a tumor, atherosclerotic tissue, or other related tissue. Embodiments of the present invention employ the natural ability of the immune system to move monocytes to areas of inflammation and also amplify it.

### Tumorous Tissue

Human tumors are often characterized by substantial heterogeneity and divergent development of subpopulations of tumor cells within the same tumor, most likely due to various somatic genetic and epigenetic alterations (Sottoriva et al., 2013). Herein, the U87MG Human Glioblastoma mouse model and the Eμ-myc/Arf-/- C57BL/6 B-cell lymphoma mouse model were used to demonstrate the utility of peptide-functionalized SWNTs to locate and to deliver treatment to tumorous tissue.

Glioblastoma (GB) is the most common primary brain malignancy, it is highly aggressive and carries a poor prognosis due to a lack of effective treatment options. The divergent development of subpopulations of cells within the same tumor is believed to be responsible for a high variation in response to treatment (Sottoriva et al., 2013). In the mouse model used, human glioblastoma was experimentally induced by transplanting U87MG human tumor cells into SCID mice.

The second mouse model, the Eμ-myc/Arf-/- transgenic mouse in a C57BL/6 background, provided a valuable model for the utility of peptide-functionalized SWNTs in locating and providing treatment to B-cell lymphomas. Eμ-myc transgenic mice bear the cellular myc oncogene coupled to the immunoglobulin μ enhancer and develop a fatal lymphoma within a few months of birth (Mori S et al. 2008; Adams, 1985). In Eμ-myc/Arf-/- transgenic mice, the Arf-gene is inactivated. The Arf-gene is a tumor suppressor and counteracts lymphomagenesis in Eμ-Myc mice. However, when the Arf-gene is inactivated, which occurs in 25% of Eμ-myc transgenic mice naturally, the Eμ-Myc-induced development of lymphoma is accelerated (Bertwistle and Sherr (2007).

When the single-walled carbon nanotubes have been functionalized with a peptide specific for the vasculature in the targeted tissue, these monocytes move from the blood flow towards the blood vessel inner surface (the endothelium), ultimately interacting with this surface, moving along the surface and into a tumor, atherosclerotic tissue, and possibly other diseased tissue. This allows specific visualization as well as localization and delivery of treatment to the diseased tissue, where the monocytes and ultimately macrophages carrying the SWNTs gather.

In various embodiments of the invention, the single-walled carbon nanotubes have been functionalized with RGD peptides, which appeared to guide the monocytes to tumorous tissue. While the conjugation of SWNTs to an RGD peptide delayed their uptake into monocytes, it was found to markedly increase (p<0.0001) the targeting of SWNT-loaded cells to the tumor. Furthermore, conjugation with a RGD-sequence containing peptide encouraged increased interaction of the Ly-6C^{hi} monocytes with vascular endothelium and resulted in a rise in macrophages at the tumor site due to enhanced SWNT delivery.

As described in Example 1, one embodiment uses a mouse having an implanted tumor, and the ability of the SWNT-laden monocytes to locate to, and congregate in, the tumor. As a result, the tumor is found to exist, and its location and size can be determined. Using this information, related SWNTs carrying therapeutics can be delivered to the tumor site to stop progression of the tumor, or to partially or completely eliminate the tumor. Thus, in this case, the therapeutic is directed to destruction, or limiting the adverse activity (for example, via re-direction of the differentiation of the monocyte), of the detected tumor tissue.

In other embodiments, circulating Ly-6C monocytes are shown to selectively pick up SWNTs and infiltrate the tumor mass in the tumor murine models. Unlike the Ly-6C^{hi} monocytes in blood and spleen, the tumor monocytes continued to accumulate SWNT even at 24 hours after i.v. injection of SWNTs.

### Anti-Tumor Therapeutics

Therapeutics which are contemplated in the context of the present invention to be delivered to tumorous tissue for treatment thereof, using single-walled carbon nanotubes that may be conjugated to a peptide such as RGD, include but are not limited to agents that cause DNA damage such as alkylating agents such as cyclophosphamide, mechlorethamine, uramustine, melphalan, chlorambucil, ifosfamide, bendamustine, carmustine, lomustine, streptozocin, busulfan or alkylating-like, platinum based agents such as cisplatin, carboplatin, nedaplatin, oxaliplatin, satraplatin, triplatin tetranitrate.

Further contemplated anti-tumor therapeutics include agents that inhibit RNA or DNA synthesis such as anthracyclines which are represented by daunorubicin, doxorubicin, epirubicin, idarubicin, valrubicin, mitoxantrone. Anti-tumor therapeutics also encompass cytoskeletal disruptors such as paclitaxel and docetaxel as well as epothilones such as patupilone, sagopilone and ixabepilone; inhibitors of topoisomerase I such as irinotecan and topotecan and inhibitors of topoisomerase II such as etoposide, teniposide, tafluposide; nucleotide analogs and precursor analogs such as azacitidine, azathioprine, capecitabine, cytarabine, doxifluridine, fluorouracil, gemcitabine, hydroxyurea, mercaptopurine, methotrexate, tioguanine; peptide antibiotics such as bleomycin and actinomycin; protein kinase and proteasome inhibitors including bortezomib, erlotinib, gefitinib, imatinib, sunitinib, vemurafenib, vismodegib; salinosporamide A, carfilzomib; all-trans retinoic acid and retinoids such as tretinoin, isotretinoin, alitretinoin, bexarotene; vinca alkaloids and derivatives including vinblastine, vincristine, vindesine, vinorelbine (Nefedova et al, 2007); synthetic triterpenoids such as CDDO-Me (Nagaraj et al., 2010);

Monoclonal antibodies to inhibit tumor growth are contemplated herein as well including agents such as cetuximab, panitumab, rituximab, bevacizumab, ipilimumab, ofatumumab, ocrelizumab.

### Atherosclerotic Tissue

The approach outlined above for tumorous tissue can also be translated into locating and treating atherosclerotic tissue. Ly-6C^{hi} monocytes have been shown to be involved in atherosclerosis. (Swirski et al, 2007). These monocytes adhere to vascular endothelium, infiltrate lesions such as those formed by plaque, and became lesional foamy macrophages. The macrophages release all sorts of proteases such as metalloproteases, pepsin and other destructive molecules that attack the extracellular matrix. If left untreated, these macrophages will create holes in the blood vessel endothelium and cause major damage. Because of this affinity to atherosclerotic tissue, the procedures described in the following examples using peptide-conjugated nanotubes or plain nanotubes to locate and treat tumors are directly applicable to atherosclerosis. The Ly-6C^{hi} monocytes provide not only a diagnostic tool for atherosclerosis, but also a unique method for delivering treatment to this tissue.

Plain or peptide-conjugated SWNTs are injected into the blood stream. In mice, the resulting SWNT-Ly-6C^{hi} monocytes or RGD-SWNT-Ly-6C^{hi} monocytes move then towards atherosclerotic tissue along the endothelium, in addition to moving toward any existing tumors, and accumulate there. Detecting diseased tissue is accomplished by locating the accumulated SWNTs in the Ly-6C^{hi} monocytes in mice or CD14+ monocytes in humans. Delivering treatment to diseased tissue is accomplished by attaching therapeutics to plain SWNTs or peptide-conjugated SWNTs before the SWNTs are administered and accumulate in the diseased tissue.

For the studies herein, a murine atherosclerotic model was used, wherein the mice were fed a high-fat diet for 30 days and diabetes was induced by 5 daily intraperitoneal injections of streptozotocin. The formation of atheroma plaques was induced by the ligation of one carotid artery (left artery ligated below the bifurcation), while the other non-ligated artery was used as a control.

### Anti-Atherosclerotic Therapeutics

Therapeutics which are contemplated in the context of the present invention to be delivered to atherosclerotic tissue for treatment thereof, using single-walled carbon nanotubes that may be conjugated to a peptide such as RGD, include various types of lipid lowering agents including statins such as simvastatin, pitavastatin, pravastatin, rosuvastatin, lovastatin, fluvastatin, atorvastatin; fibrates such as bezafibrate, ciprofibrate, clofibrate, gemfibrozil, fenofibrat; inhibitors of the cyclooxygenase-2 pathway such as celecoxib; rofecoxib; inhibitors of the arachidonate 5-lipoxygenase pathway such as zileuton, minocyline; bile acid sequestrants such as colestipol, cholestyramine; Niacin; Probucol; lysophosphatidic acid antagonists; acyl CoA: cholesterol acyltransferase inhibitors.

### Treatment by Delivering Therapeutics or Radiation Using SWNTS

SWNTs exposed to laser light in the near-infrared range (700-1100 nm) have been shown to induce thermal destruction and, thus, can be used for thermal destruction of tumor or atherosclerotic cells (Robinson et al., 2010; Gannon et al., 2007; Kosuge et al., 2012).

Therapeutics can be delivered to a tumor or to atherosclerotic tissue. For the former, chemotherapeutic drugs can be delivered by attachment to the SWNTs. Examples of such drugs include, but are not limited to, alkylating agents such as cisplatin and cyclophosphamide, anti-metabolites such as mercaptopurine, plant alkaloids and terpenoids such as taxanes and vincristine, topoisomerase inhibitors such as irinotecan, cytotoxic antibiotics such as actinomycin and doxorubicin, etc. Doxorubicin can be stacked on carbon nanotubes for use as a chemotherapeutic (Zhuang et al., 2009). Nanoparticles have also been found useful for delivering poorly soluble drugs such as paclitaxel.

For treatment of atherosclerosis, various drugs can be attached to the SWNTs. Some examples include, but are not limited to, anti-proliferatives, anti-mitotic drugs, anti-platelets, anti-inflammatory drugs such as dexamethasone and estradiol, anti-thrombotics, thrombolytics, cytotoxic drugs and cytostatic drugs. Dosage of the drugs is determined by factors such as weight and size of the mammalian subject in need of the drug and solubility of the drug.

### Experimental Procedures

The following examples show, through intravital microscopy, fluorescence activated cell sorting (FACS) and Raman imaging, the specificity of the peptide-SWNT-Ly-6C^{hi} monocyte interaction in mice, the movement of the particular SWNT-monocytes, and the entry of the SWNT-laden monocytes into a tumor. In one particular embodiment of the invention, the SWNTs have been functionalized with RGD peptides, which appear to guide the monocytes (and macrophages that the monocytes turn into) to a tumor. This embodiment, as described in Example 1, uses a mouse having an implanted tumor, and the ability of the SWNT-laden monocytes to locate to, and congregate in, the tumor. As a result, the tumor is found to exist, and its location and size can be determined. Using this information, related SWNTs carrying therapeutics can be delivered to the tumor site to stop progress of the tumor, or to partially or completely eliminate the tumor. Thus, in this case, the therapeutic is directed to destruction, or limiting the adverse activity, of the detected tumor tissue.

The following experiments use the materials and methods described below.

### Animal Use

Animal experiments were conducted in compliance with all relevant guidelines and regulations, and were approved by the Stanford Administrative Panel on Laboratory Animal Care (APLAC).

8 wks to 6 months old male BALB/c, FvB, CB. 17, C57BL/6, and SCID transgenic mice (Charles Rivers or Jackson Laboratories) were housed at Stanford Research Animal Facility (RAF) under Stanford Institutional Animal Care and Use Committee (IACUC) protocols. Mice were monitored visually, ensuring no outward signs of distress.

Balb/c Mice.

Mice were injected with human EGFP-transfected U87MG tumor cells and the tumor was allowed to grow for about 10-14 days. EGFP is enhanced green fluorescent protein, first isolated from jellyfish, and then modified to enhance the green fluorescence.

Scid Mice.

Scid mice were orthotopically implanted with U87MG human glioblastoma cell lines and the tumor was allowed to grow for about 14 days.

Eu-myc/Arf-/- C57BL/6 transgenic mice bear the cellular myc oncogene coupled to the immunoglobulin μ enhancer and have an inactivated Arf-gene; they develop a fatal lymphoma within a few months of birth as well as tumors in spleen and bone marrow.

FvB Mice.

As already described above, 8 wk-old male FVB mice were fed a high-fat diet and diabetes was induced by 5 daily intraperitoneal injections of streptozotocin (STZ; 40 mg/kg). The left carotid was then ligated and after 2 weeks the left, diseased artery developed atheroma plaques and was harvested for hi-D FACS analysis. The right, healthy artery that had not been ligated was also harvested and used as control.

### Preparation of SWNTS

HiPco single-walled carbon nanotubes were obtained from Carbon Nanotechnologies Inc. Poly(maleic anhydride-alt-l-octadecene) (molecular weight 30 to 50 kDa) was purchased from Sigma-Aldrich (St. Louis, Mo.). Both mPEG-NH2 and DSPE-mPEG were obtained from Laysan Bio Inc. Regenerated cellulose dialysis membrane bags were obtained from Fischer Scientific.

### Synthesis of C18-PMH-MPEG

Polymer C18-PMH-mPEG was synthesized in the following manner. Methoxy-poly(ethylene glycol)-amine (285.7 mg, 0.05714 mmol, mPEG-NH2, 5 kDa) was combined with poly(maleic anhydride-alt-l-octadecene) (10 mg, 0.0286 mmol) in 15 mL of a 9:1 DMSO/pyridine mixture. The solution was allowed to stir for 12 h at room temperature, followed by the addition of 1-ethyl-3-(3-dimethylaminopropyl) carbodiimide hydrochloride (21.8 mg, 0.11 mmol) (EDC.HCl). The reaction was continued for 24 h, followed by dialysis to remove excess mPEG-NH2.

### Preparation of SWNT Suspensions

A 50% DSPE-mPEG/50% C18-PMH-mPEG SWNT nanotube solution was prepared by combining 0.2 mg/mL of HiPco tubes with 0.6 mg/mL of DSPEmPEG and 0.6 mg/mL of C18PMH-mPEG in 30 mL of water. The solution was sonicated for 1 h followed by centrifugation (6 h, 22 000 g) to remove any bundles or aggregates. The resulting supernatant was collected and filtered eight times through a 100 kDa pore size filter (Millipore) to remove excess polymer. 200 μL solutions of 2 μmol/L SWNT were prepared in 2×phosphate-buffered saline (PBS). This was done by adjusting the concentration based on the absorption peak at 808 nm having an extinction coefficient [5] of 7.9×106 L/mol cm.

### Functionalization of SWNTS

Thiolated RGD or RAD peptide was used directly. The thiolated peptide was protected from oxidation by adding EDTA to prevent heavy metal-catalyzed oxidization during the conjugation with nanotubes. Maleimide groups were introduced onto SWNTs by reacting PL-PEG-amine functionalized SWNTs with a sulfosuccinimidyl 4-N-maleimidomethyl cyclohexane-/-carboxylate (Sulfo-SMCC) bifunctional linker. The activated SWNTs were then reacted with thiolated RGD or RAD peptides, obtaining targeted SWNT bioconjugates (Liu et al., 2009). Cy5.5 fluorescent dye (GE Healthcare, Piscataway, N.J.) was also conjugated to the SWNTs to make them visible through a fluorescent microscope.

### Observing SWNTs

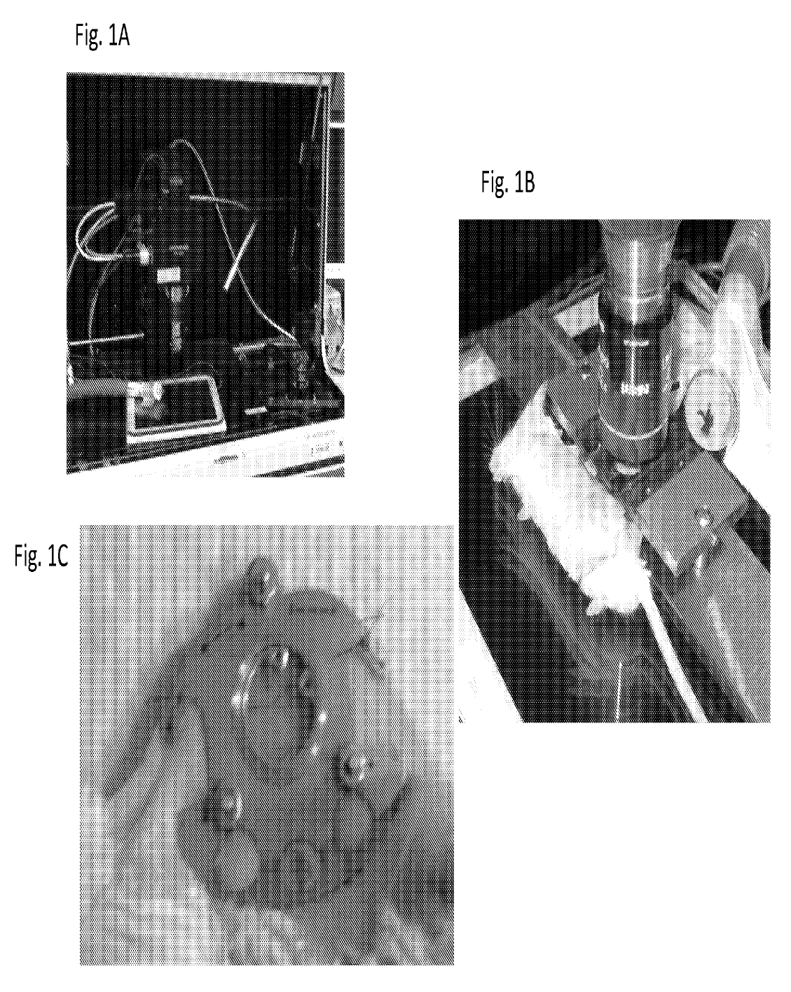

To observe nanotube targeting and cell uptake in living subjects, intravital microscopy was performed using an IV-100 fluorescence-based instrument, as shown in FIGS. 1a and 1b. The microscope employs four input lasers and three simultaneous output channels to dynamically image tissue such as a tumor in a living subject. A magnified picture of the lens and the animal is shown in FIG. 1b. FIG. 1c shows the animal model used. It is a dorsal skinfold titanium chamber (medium sized kit from APJ Trading, Los Angeles, Calif.) with a window diameter of about 12 mm, which is surgically. (Li et al., 2004). This device allows stabilization of mouse motion and provides a transparent window for optical microscopy.

While imaging of the RGD-SWNT-Ly-6C^{hi} monocytes has been performed here using fluorescent dyes, other means of imaging are also available. In some embodiments of the present invention, fluorescent dyes were added to the SWNTs, making them imageable in a fluorescence based intravital microscopy. Some intrinsic methods include, but are not limited to, Raman imaging, photoacoustics (de la Zerda et al., 2010) and near-infrared (NIR) detection (Welsher et al., 2009). Other techniques include, but are not limited to, placing Gadolinium inside the SWNTs and imaging with MRI, radiolabeling the SWNTs, and adding Iodine to the SWNTs to make them imageable via CT or X-ray.

### FACS—Fluorescence Activated Cell Sorting

Cell suspensions were pre-incubated with anti-CD16/CD32 mAb to block FcγRII/III receptors and stained on ice for 30 min. with the following fluorochrome-conjugated mAb in a 12-color staining combination: FITC-Ly-6C (monocyte marker), PE-CD62L (lymph node homing marker), PECy5-CD5 (T cell marker), PECy5.5-CD19 (B cell marker), PECy7-Gr-1 (granulocyte marker), APC-CD49b (NK cell marker), APCCy7-CD11b (myeloid marker); Pacific Blue-F4/80 (macrophage marker), Biotin-CD11c (dendritic cell marker), Biotin or PE-MHC-II (antigen presentation marker), Biotin or APC-CD80/CD86 (activation markers), Propidium Iodide (PI, discriminate live from dead cells). Cells were then washed and stained again on ice for 15 min. with streptavidin Qdot 605 (Invitrogen) to reveal biotin-coupled antibodies. Antibodies were either purchased (Invitrogen and BD Pharmingen) or conjugated in our laboratory. After washing, stained cells were resuspended in 10 μg/mL PI, to exclude dead (i.e., PI-negative) cells. Cells were analyzed or sorted on Stanford Shared FACS Facility instruments (Becton Dickinson LSRII or FACSAria). Data were collected for 0.2 to 1×106 cells. Data were analyzed with FlowJo software (TreeStar). To distinguish auto-fluorescent cells from cells expressing low levels of individual surface markers, upper thresholds for auto-fluorescence were established by staining samples with fluorescence-minus-one control stain sets (See Roederer, 2001; Herzenberg et al., 2006) in which a reagent for a channel of interest is omitted.

### Example 1: Visualization of the Movement of Single-Walled Carbon Nanotubes (SWNTS)

This example shows the injection of various forms of nanotubes into the body of a mouse that has at least one tumor. Movement of the nanotubes is followed by the use of fluorescent dyes as visualized through intravital microscopy.

To observe nanotube targeting, fluorescence microscopy was used. This consists of the instrument shown in FIGS. 1a and 1b, with the lens juxtaposed to the mouse's body. The microscope employs four input lasers and three simultaneous output channels to dynamically image a tumor in living subjects. FIG. 1c shows the dorsal skinfold chamber which is surgically implanted into the mice; the device allows stabilization of mouse motion and provides a transparent window for optical microscopy.

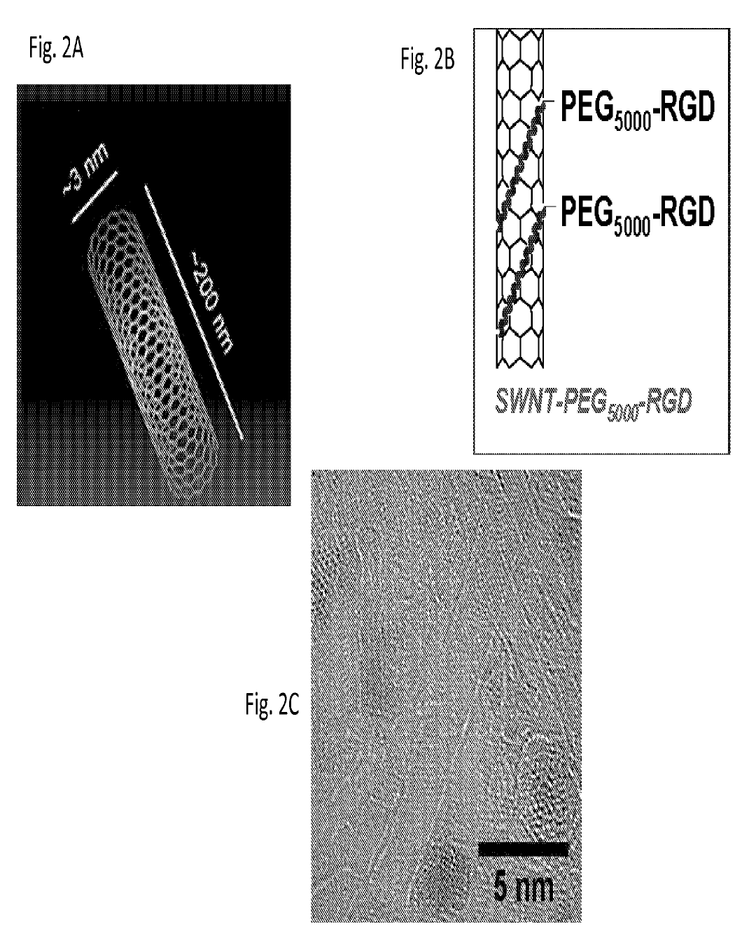

FIG. 2 shows nanotubes in a variety of forms. FIG. 2a shows the netting-type shape of the nanotubes used which have dimensions that range from approximately 3 nm to about 200 nm. FIG. 2b illustrates the attachment of the peptide RGD to a SWNT via a PEG 5000 linker, to which the peptide is conjugated. FIG. 2c shows an electron micrograph of a SWNT having dimensions similar to the ones used in this work.

### Example 2: SWNTS Functionalized with Peptides

For initial intravital microscopy, mice were injected into the tail with approximately 5×10⁵ EGFP-transfected U87MG tumor cells and the tumor was allowed to grow for about 10-14 days. EGFP is enhanced green fluorescent protein, first isolated from jellyfish, and then modified to enhance the green fluorescence. Cy5.5 was used to show the nanotubes, and a long-term dye was used to show the circulating blood.

For intravital microscopy of the SWNTs, 18 mice were injected with various experimental and control nanotubes: SWNTs with conjugated RGD, SWNTs with conjugated RAD, and plain SWNTs without attached peptides, as well as BSA without any SWNTs and other controls. SWNT behavior was visualized from injection into the mouse until about 4 hours post-injection, and then at designated time-points throughout the first day and first week post-injection. At each time point, 5-20 fields-of-view in the tumor were acquired to create a time series. More than 1500 total blood vessels were analyzed.

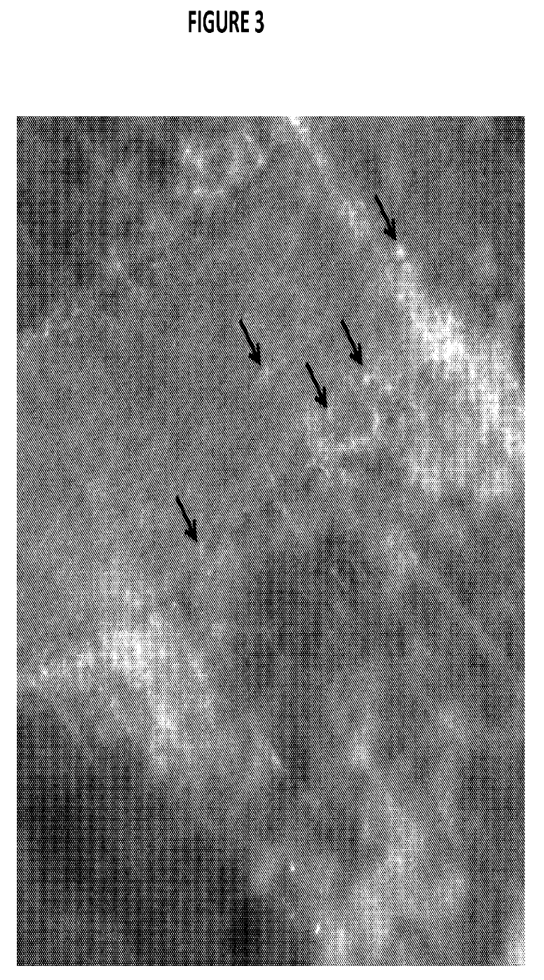

Upon injection of the RGD-SWNTs (SWNTs with conjugated RGD), there don't appear to be any circulating cells that take up the nanotubes right away. About two hours after the injection of RGD-SWNTs, circulating cells were noticeable which had taken up the RGD-SWNTs and were moving through the blood vessels, as can be seen in FIG. 3.

Interestingly, with no peptide conjugated, uptake of nanotubes into the circulating cells can be observed within a few tens of seconds after injection. This is in contrast to the time (two hours or more, as described above) it takes RGD-conjugated nanotubes to get into cells.

### Example 3: Peptide Dependency of the SWNT Uptake

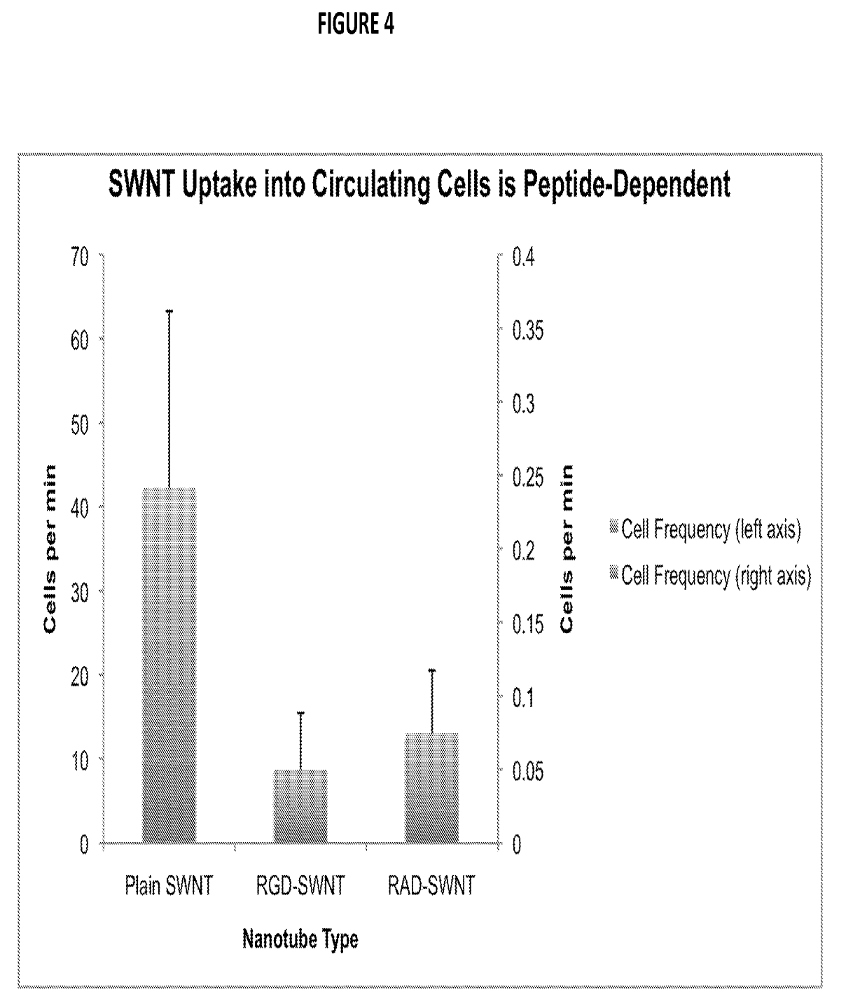

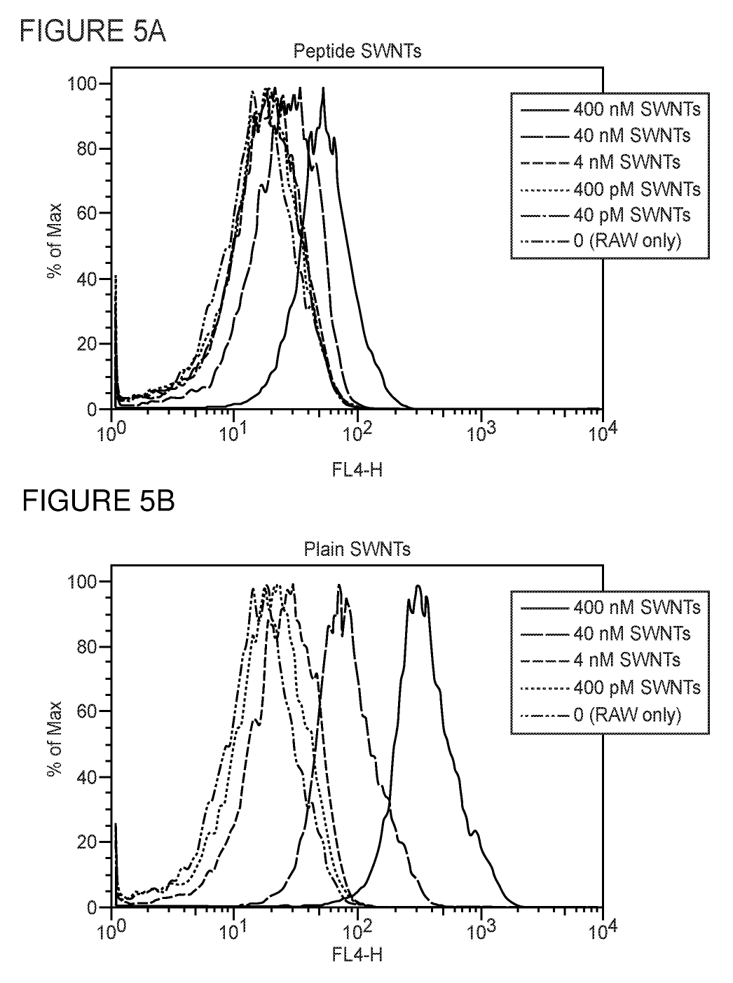

FIG. 4 illustrates the uptake of SWNTs and shows that uptake is peptide dependent. Three types of nanotubes were compared: plain, i.e. non-conjugated, SWNTs; RGD-conjugated SWNTs, and RAD-conjugated SWNTs. Cells per minute per field of view were counted within 10 minutes of injection. As seen in FIG. 4, uptake of the SWNTs is clearly a function of peptide presence (p<0.001). While the plain SWNTs were taken up into circulating cells almost immediately after injection, uptake of RGD- or RAD-conjugated SWNTs into circulating cells was much slower. The kinetics of interaction between cells containing RGD- or RAD-SWNTs and the vasculature is shown by the amount of cells per minute. Clearly, plain SWNTs are taken up by circulating cells much faster than are RGD- or RAD-conjugated SWNTs. The same type of information is shown in FIG. 5, where in vitro experiments verified the peptide dependence of uptake of SWNTs into RAW cells. At even 10× lower concentrations, more plain SWNTs entered the RAW cells than peptide-conjugated SWNTs after one hour of incubation. This can be observed, for example, when the uptake of 40 nm plain SWNTs is compared to the uptake of 400 nm peptide SWNTs.

### Example 4: Determining Type of Cells that Take Up Peptide-SWNTs

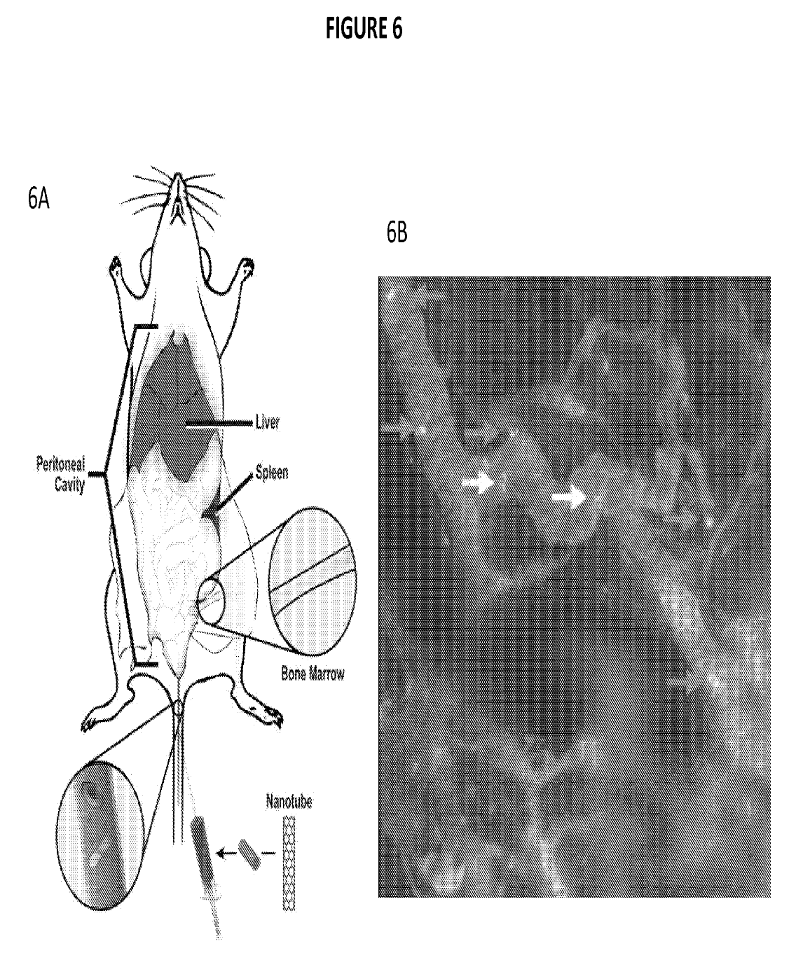

Injection of SWNTs into an animal preceded an analysis by FACS of what type of cells took up the SWNTs. FIG. 6A shows from what parts of the mouse the cell samples were taken. Liver, spleen, peritoneal cavity and bone marrow samples were studied and compared to blood samples from the mouse tail. FIG. 6B shows SWNTs moving through the vasculature and interacting with the endothelium.

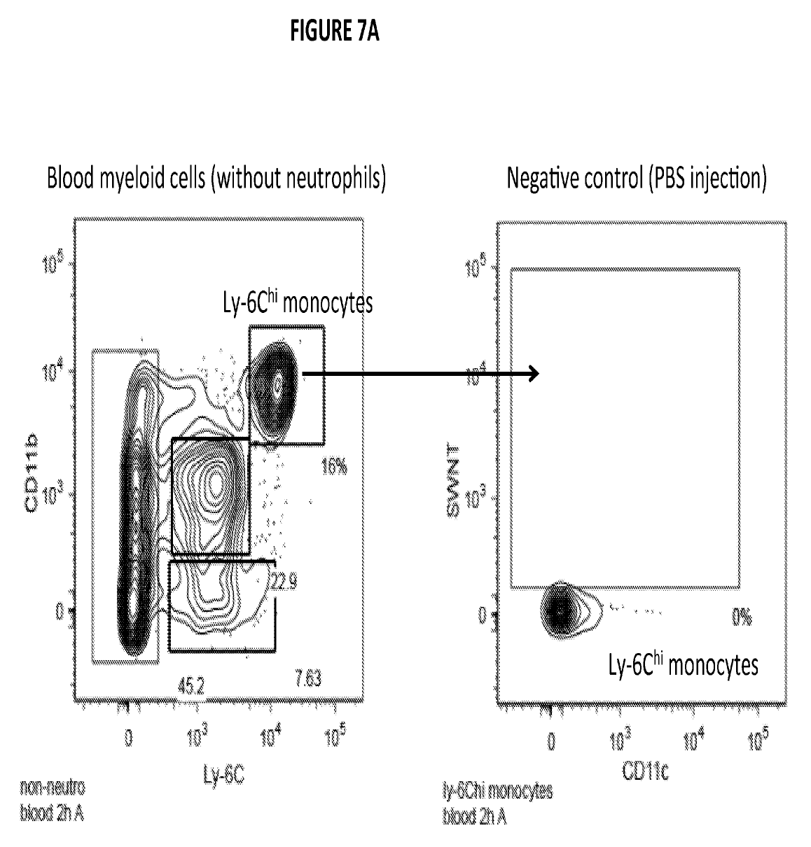

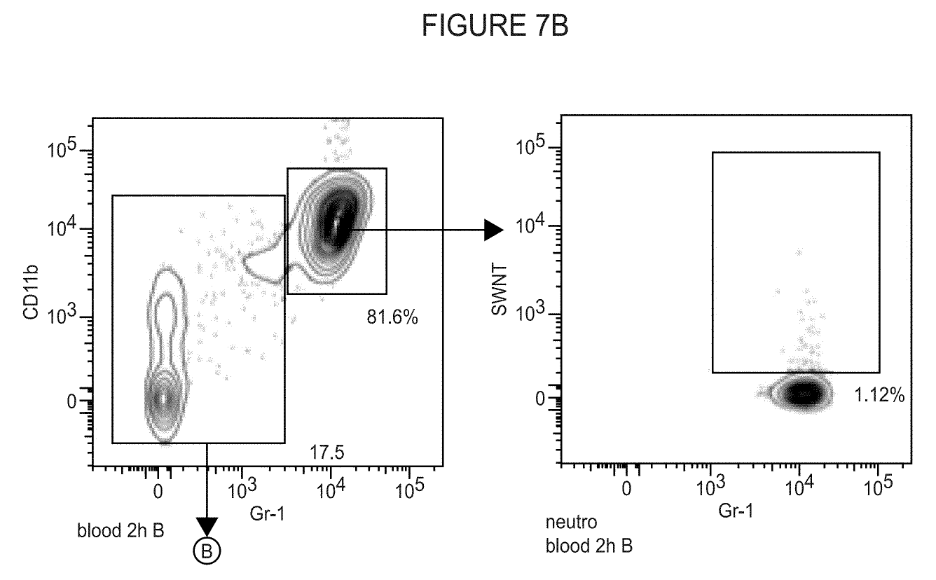

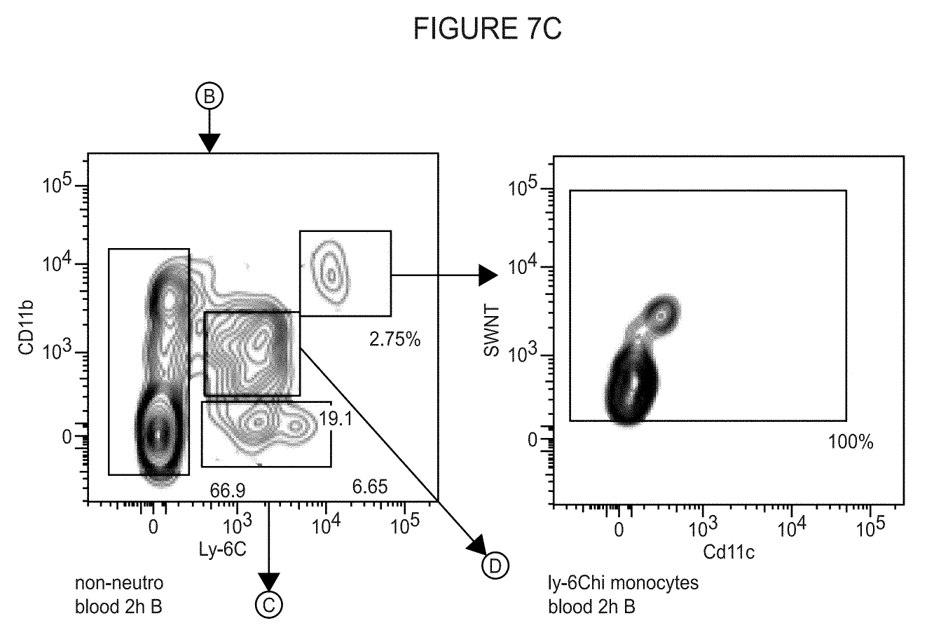

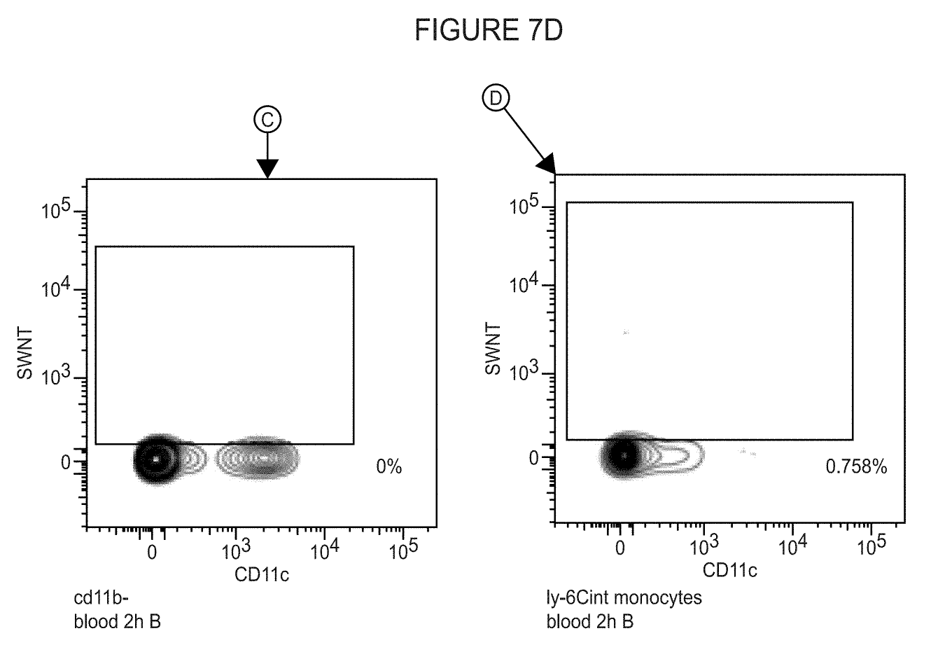

Blood cells were prepared and sorted in a fluorescence-activated cell sorter as described above. FIG. 7A shows that SWNTs were taken up close to 100% of the time by Ly-6C^{hi} monocytes. Activation of Ly-6C^{hi} monocytes by the SWNTs is shown in FIG. 7B. FIG. 7C shows the doubling of cd11b+ (marker on activated cells) expression, further indicating activation of the monocytes due to the presence of SWNTs. Meanwhile, the number of cd11b+ cells decreases in the event of SWNT injury, as can be seen in FIG. 7D.

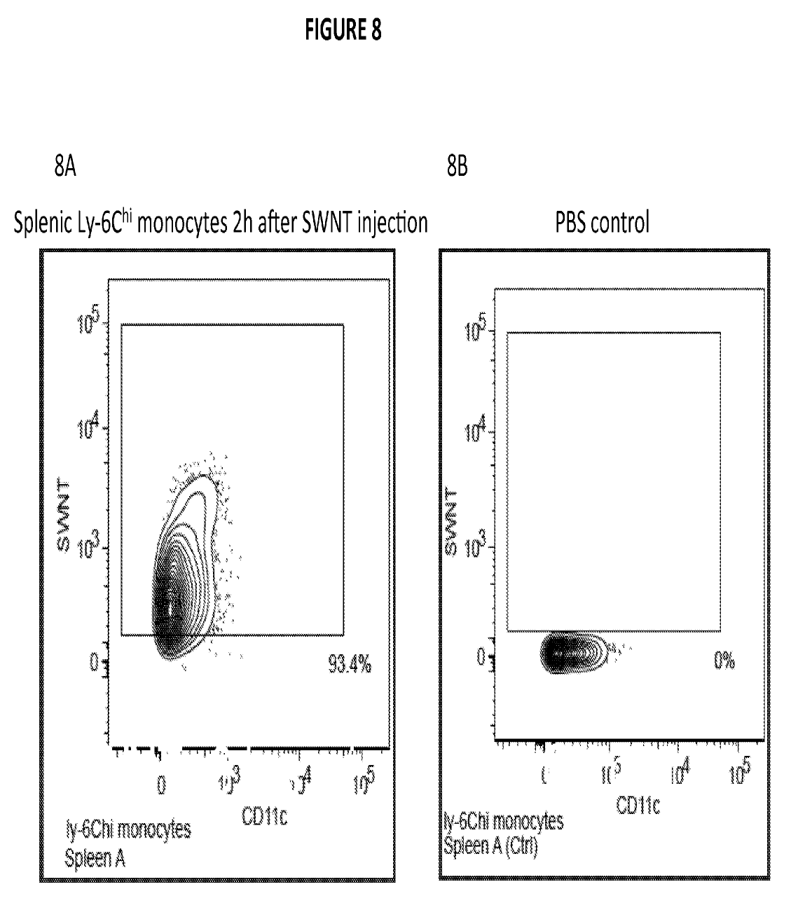

FIG. 8 shows the interaction of SWNTs with spleen cells. In the spleen Ly-6C^{hi} monocytes also take up SWNTs, as do other cells in the body. Ly-6C^{hi} monocytes from the spleen that have been exposed to SWNTs show an increase in number in FIG. 8A, while the control cells without SWNT exposure do not increase, as shown in FIG. 8B. The amount of neutrophils increased four times while the Ly-6C^{hi} monocytes increased three times over a period of about two hours. Meanwhile, the neutrophils and Ly-6C^{hi} monocytes decreased in number in the blood as their numbers increased in the spleen.

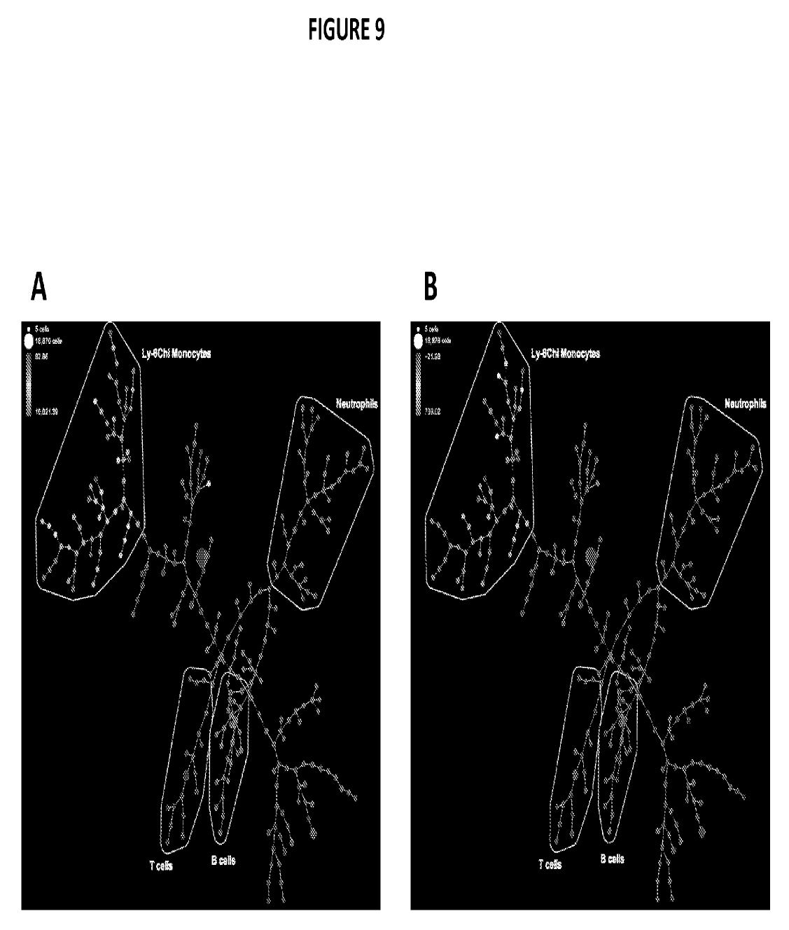

FIG. 9 illustrates the specificity of SWNTs to Ly-6C^{hi} monocytes in spleen cells, as shown by “spanning-tree progression analysis of density normalized events” (SPADE), an unsupervised computational approach to extract a hierarchy from high-dimensional flow cytometry data.

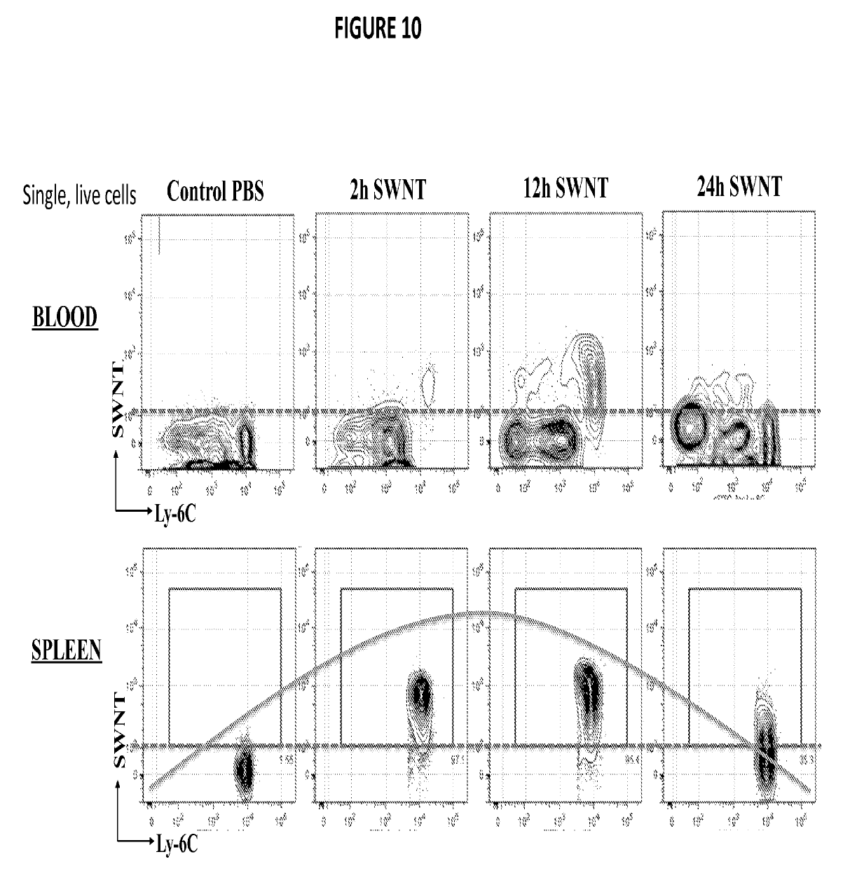

FIG. 10 shows the kinetics of SWNT internalization by immune cells in blood and spleen. Similarly to the Ly-6C^{hi} monocytes in blood, spleen Ly-6C^{hi} monocytes internalized SWNTs within two hours following injection.

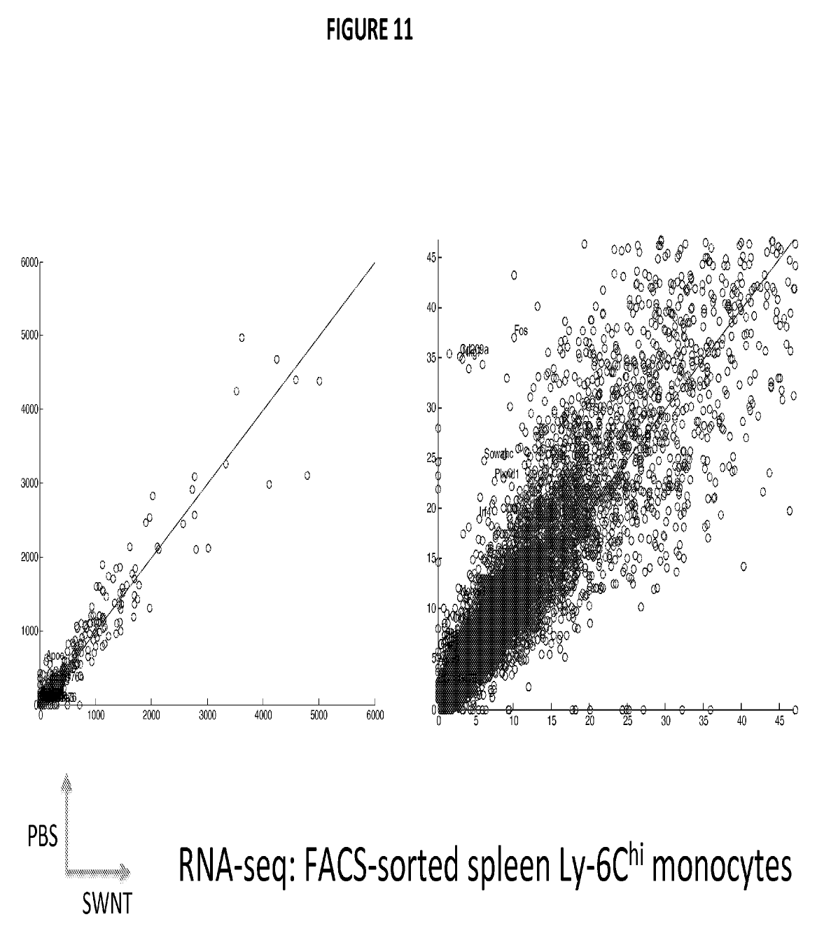

As illustrated in FIG. 11 by scatter plots of RNA-sequence data (full transcriptome analysis) for purified Ly-6C^{hi} monocytes from spleen compared to Ly-6C^{hi} monocytes from spleen control mice, both types of Ly-6C^{hi} monocytes had a similar gene expression profile.

In summary of the previous data, variables include (a) interaction of monocyte with the endothelium, (b) time, and (c) with or without peptides on the SWNTs. The following Table shows these relationships.

TABLE 1

| | | | |
|---|---|---|---|
| 1 day post | NON-INTERACTING | Plain > RAD > RGD | P = 0.0001 |
| Injection | INTERACTING | RGD > RAD > Plain | P = 0.0057 |
| >1 week | ALL INTERACTING | Plain = RAD > RGD | P < 0.0001 |
| post | | | |
| injection | | | |

This data shows that RGD, as a peptide conjugated to SWNTs and taken up by Ly-6C^{hi} monocytes, encourages interaction of the cells with the blood vessel endothelium. This may increase monocyte uptake into the tumor, as at more than one week after injection, the RGD-conjugated SWNTs in the monocytes is lower in the vasculature than are either of the other two types of SWNT conjugates. The free-flowing monocytes do not interact with the endothelium, while those cells interacting with the endothelium move along the endothelium.

### Example 5: Interaction with Tumor

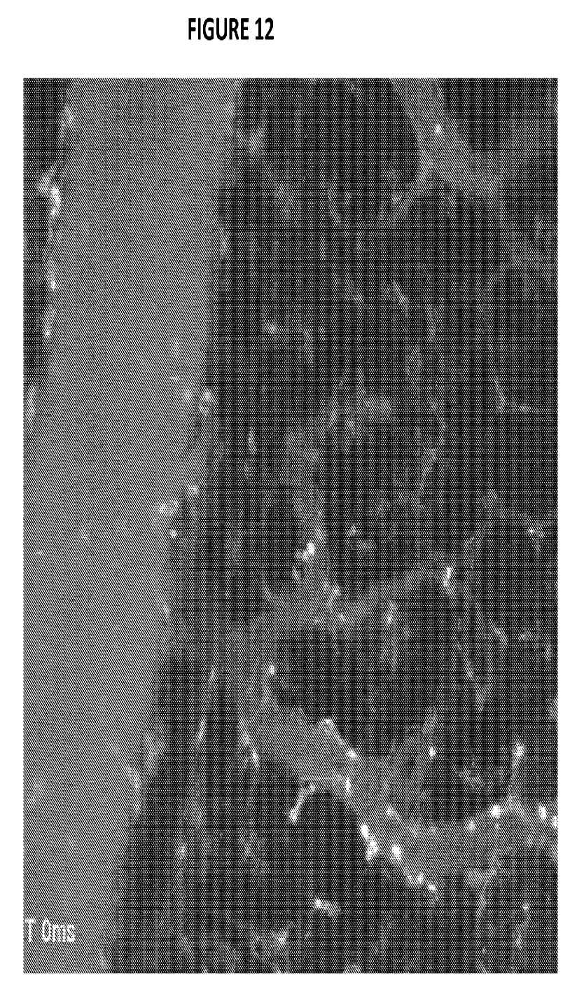

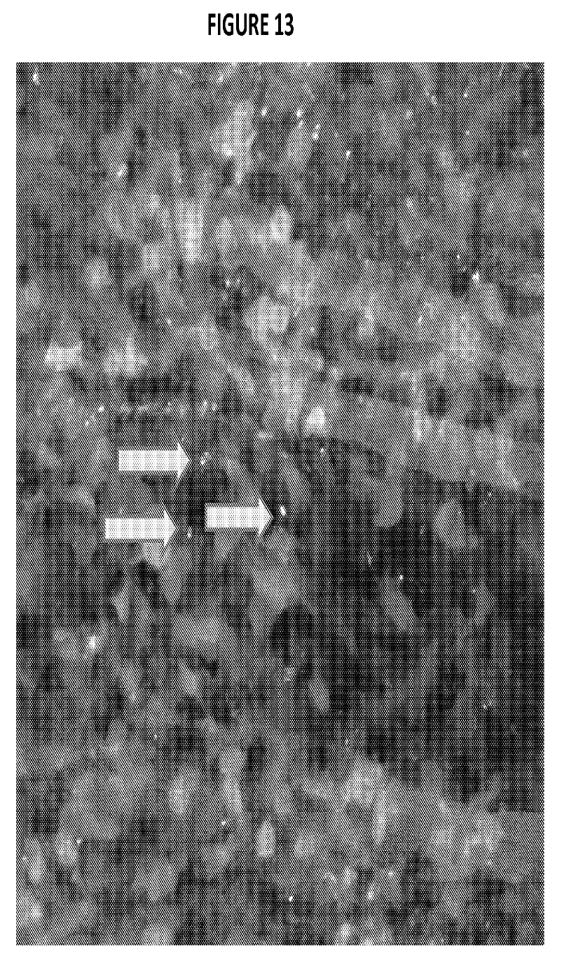

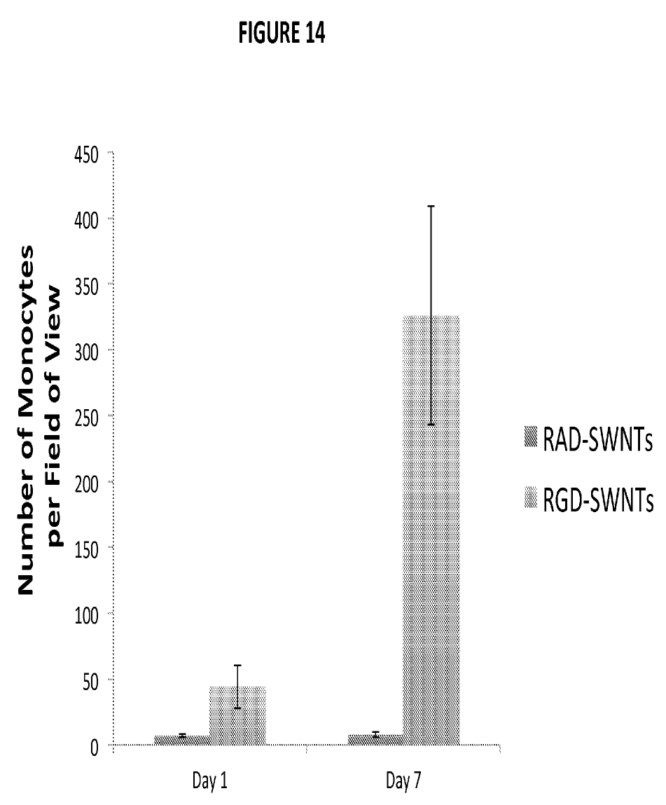

FIG. 12 is a merged view of tumor, blood vessels and SWNTs. The SWNTs appear as small dots, as indicated by the arrows. Most of the SWNT-monocytes are located along the endothelium of the blood vessels. FIG. 13 shows a later photo where the SWNT-monocytes are now appearing mainly in the tumor. FIG. 14 shows the peptide dependence of tumor targeting. The chart tallies the number of Ly-6C^{hi} monocytes conjugated to RAD (RAD-SWNTs, in red) and Ly-6C^{hi} monocytes conjugated to RGD (RGD-SWNTs, in black). The conjugation to the RGD peptide clearly caused a marked increase (p<0.0001) in targeting of SWNT-loaded cells to the tumor.

### Example 6: Population of Cells in a Tumor

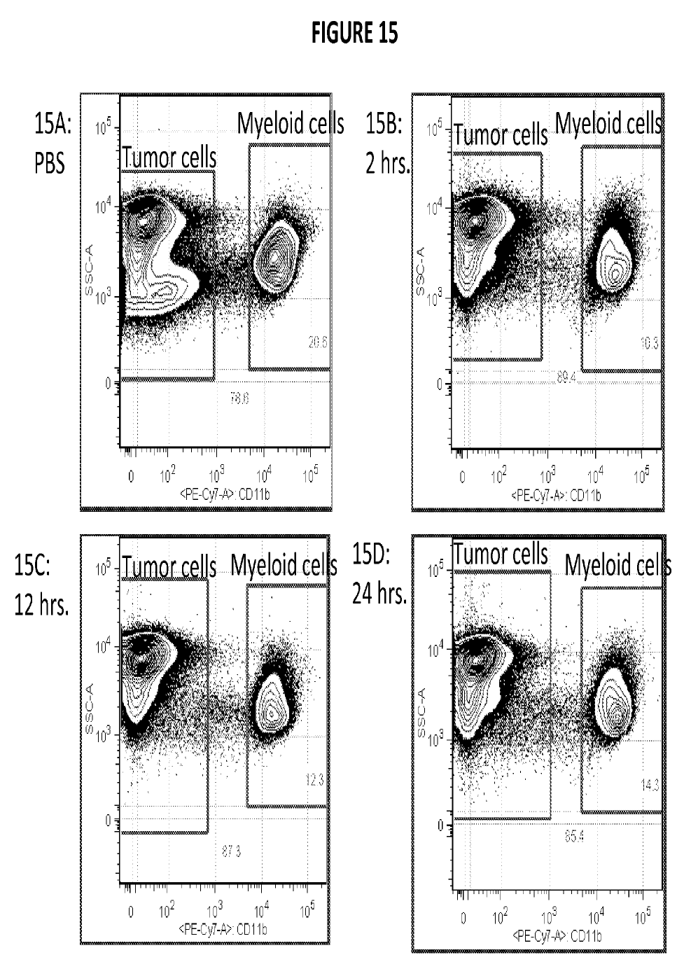

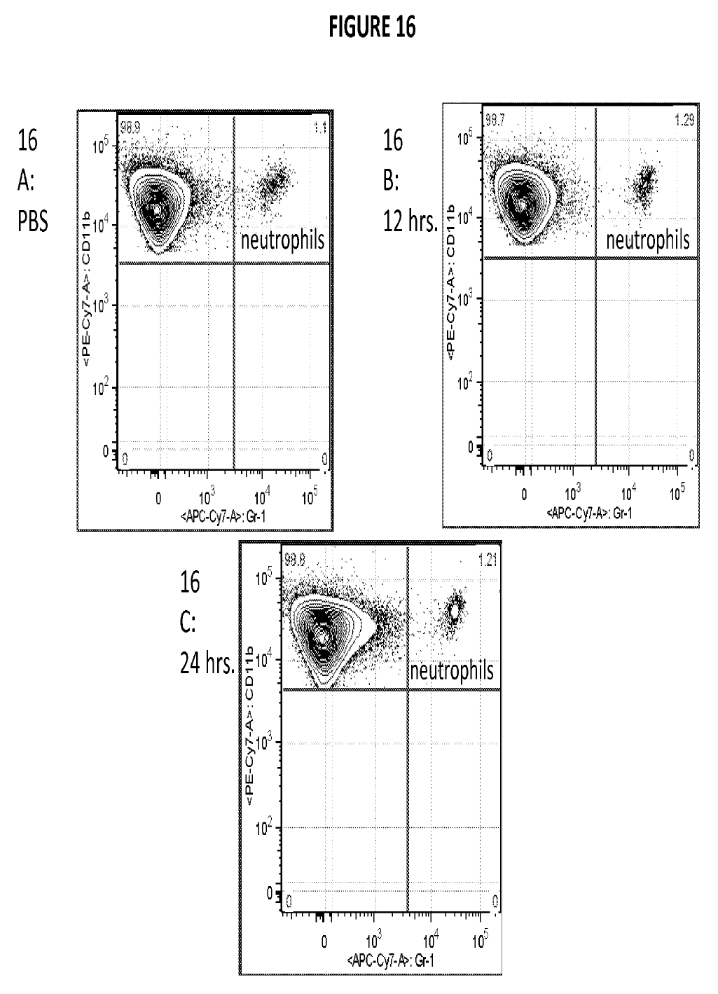

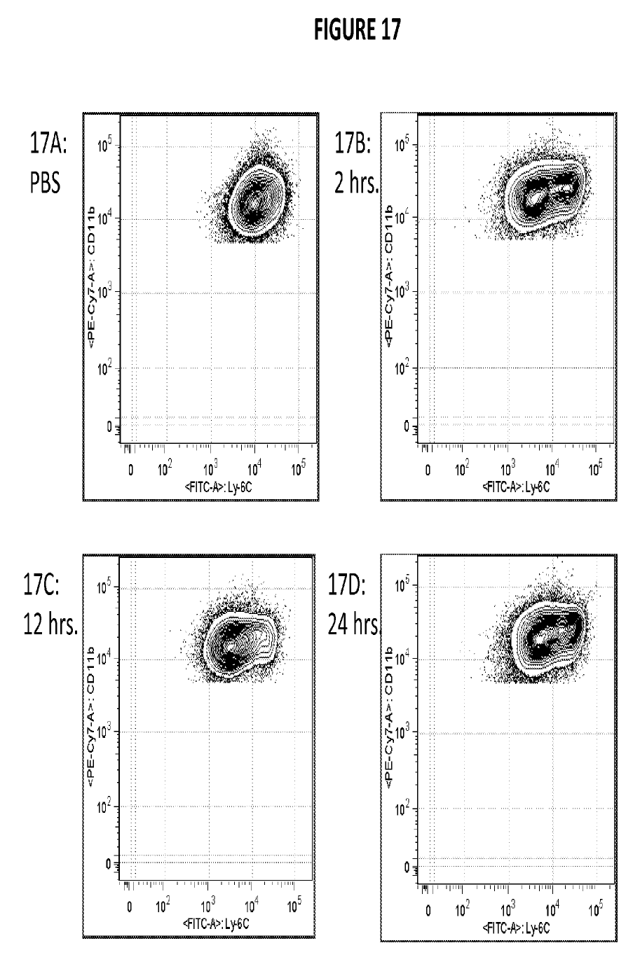

Using FACS analysis, the different types of cells within the tumor and their composition were investigated. In FIG. 15, it can be seen that myeloid cells make up 10-20% of the population at each of the time points (myeloid cells appear in the right side of each plot). The FACS data in FIG. 16 show that there are very few neutrophils in the tumor. This is unusual because normally in blood, neutrophils outnumber other immune cells; at least 70% of normal blood cells are neutrophils. This brings up the question of whether SWNTs affect monocytes in the tumor. As shown in the FACS results in FIG. 17, SWNTs induce lower Ly-6C^{hi} expression. The monocytes appear in two populations. One possibility is that the SWNTs may encourage differentiation of the monocytes.

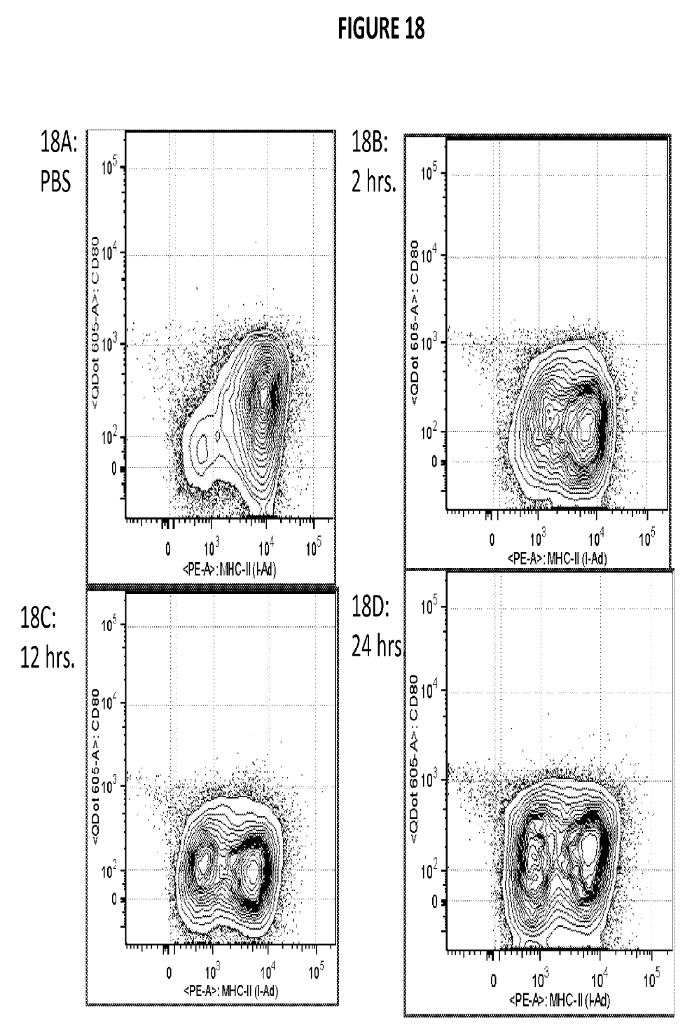

FIG. 18 shows that SWNTs do have an effect on activation of other cells. SWNTs have a negative effect on expression of both MHCII (which appear on only 3 types of cells: macrophages, dendritic cells and B cells) and CD80 expression. These effects may be due to delay or inhibition of expression. MHCII and CD80 are co-stimulatory T cell activators, and SWNT presence may lead to decreased stimulatory activity toward T cells.

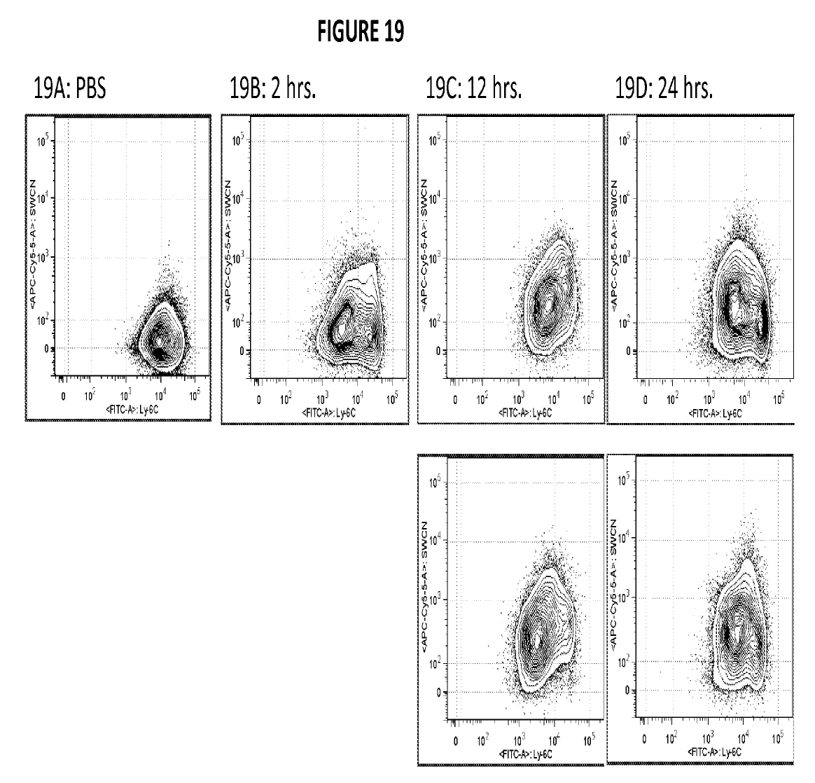

FIG. 19 shows the longitudinal progression of Ly-6C^{hi} monocytes. Over the period of one day, SWNT-laden Ly-6C^{hi} monocytes enter the tumor and begin to differentiate.

### Example 7: Ly-6C^{hi} Monocytes Exclusively Pick Up SWNTs, Providing Unique Diagnostic and Therapeutic Tools for Tumorous Cells

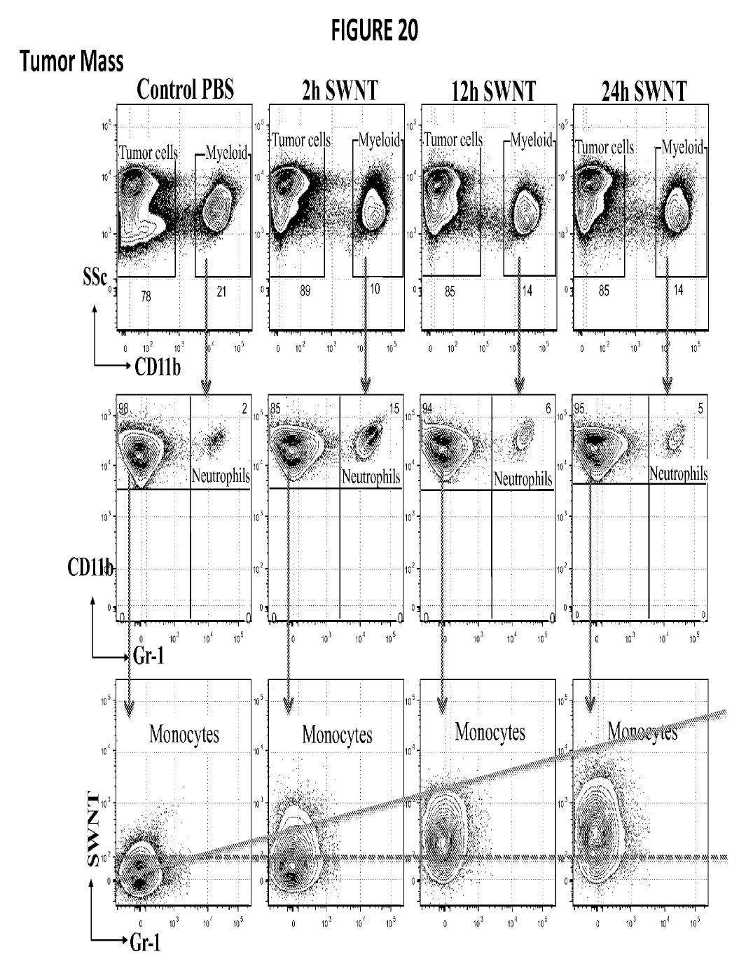

Expanding on the above described results, it could be shown that Ly-6C^{hi} monocytes selectively picked up SWNTs and infiltrated the tumor mass in murine models of the human glioblastoma (FIG. 20) and the B-lymphoma.

Once the tumor was assessed to be established in the mice, these diseased mice were injected with single-wall carbon nanotubes in comparison to control mice which carried the same disease, but were injected with PBS instead of SWNTs. After 2, 6, 12 and 12 hours groups of mice were sacrificed, tumors were separated and processed into single cell suspensions representing blood, spleen, bone marrow, liver and peritoneal cavity and analyzed by Hi-D FACS using the following parameters simultaneously: Ly-6C, I-A/I-E, CD5, CD19, CD11b, Gr-1, CD45, SWNT-Cy55, Propidium Iodide to discriminate live from dead cells, CD80/CD86, Forward and Side Scatter to determine size and granularity, respectively, NK1.1, CD49b, and F4/80.

The top panels in FIG. 20 show that about 10-20% of total tumor mass represented myeloid cells (CD11b+). Center panels show that about 2-15% of the tumor myeloid cells represented neutrophils (Gr-1^{hi}). Bottom panels show that Ly-6C^{hi} monocytes internalized SWNT in a time-dependent manner. Unlike the Ly-6C^{hi} monocytes in blood and spleen, the tumor monocytes continued to accumulate SWNT even at 24 hours after i.v. injection of SWNT.

### Example 8: LY-6C^{hi} Monocytes and Foamy Macrophages Exclusively Pick Up SWNTs, Providing Unique Diagnostic and Therapeutic Tools for Atherosclerosis

Ly-6C^{hi} monocytes have been shown to be involved in atherosclerosis (Swirski et al., 2007). These Ly-6C^{hi} monocytes adhere to vascular endothelium and infiltrate lesions such as those formed by atheromatous plaque, becoming lesional foamy macrophages (Swirski et al., 2007). These macrophages release metalloproteases, pepsins and several other damaging molecules that attack the extracellular matrix. If not treated, these macrophages create holes in the blood vessel endothelium and cause major damage. Therefore, Ly-6C^{hi} monocytes, which are known to give rise to foamy macrophages in atheromatous plaques, offer not only a diagnostic tool for atherosclerosis, but also represent a unique target for supplying treatment to atherosclerotic tissues.

The process of homing and detecting single-walled carbon nanotubes (SWNTs) in atherosclerosis is very similar to that described previously for homing and detecting SWNTs in tumors. RGD-conjugated or non-conjugated SWNTs are delivered into the blood stream. Ly-6C^{hi} monocytes in the blood stream (or elsewhere) take up SWNTs and the resulting SWNT-Ly-6C^{hi} monocytes infiltrate into the diseased artery and accumulate in atheromatous plaques (in addition to infiltrating any existing tumors). Alternatively, SWNTs may directly infiltrate the atheromatous plaques through the vascular endothelium and will then be internalized in situ by resident foamy macrophages and Ly-6C^{hi} monocytes.

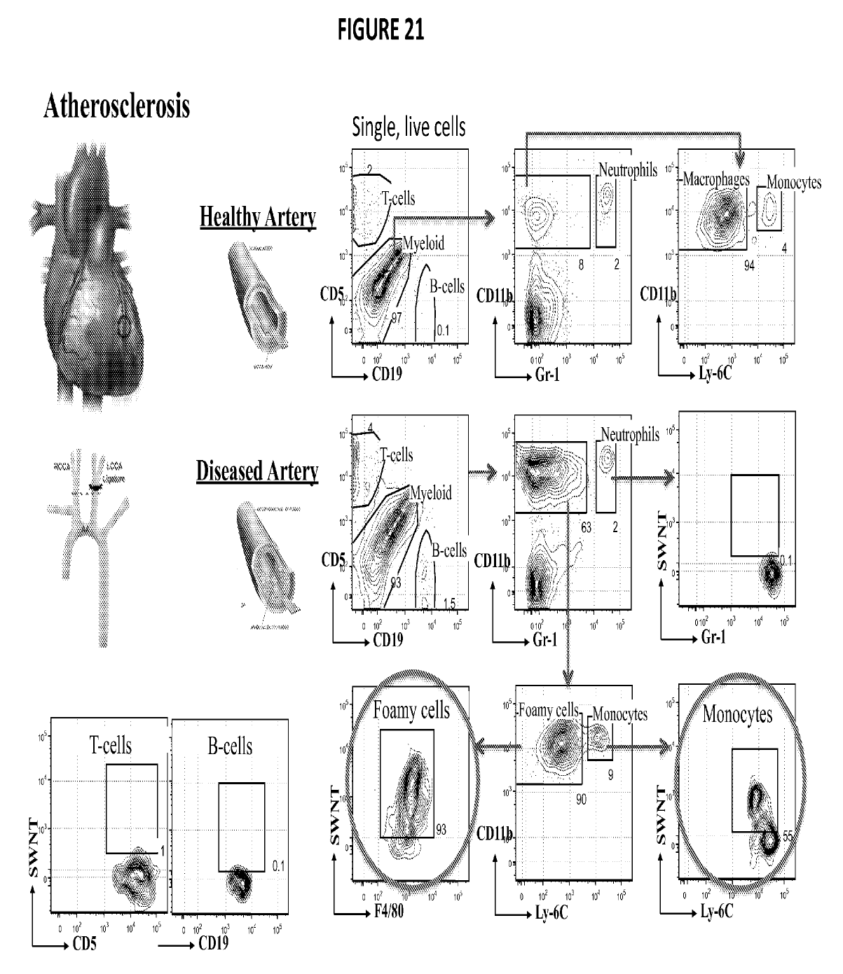

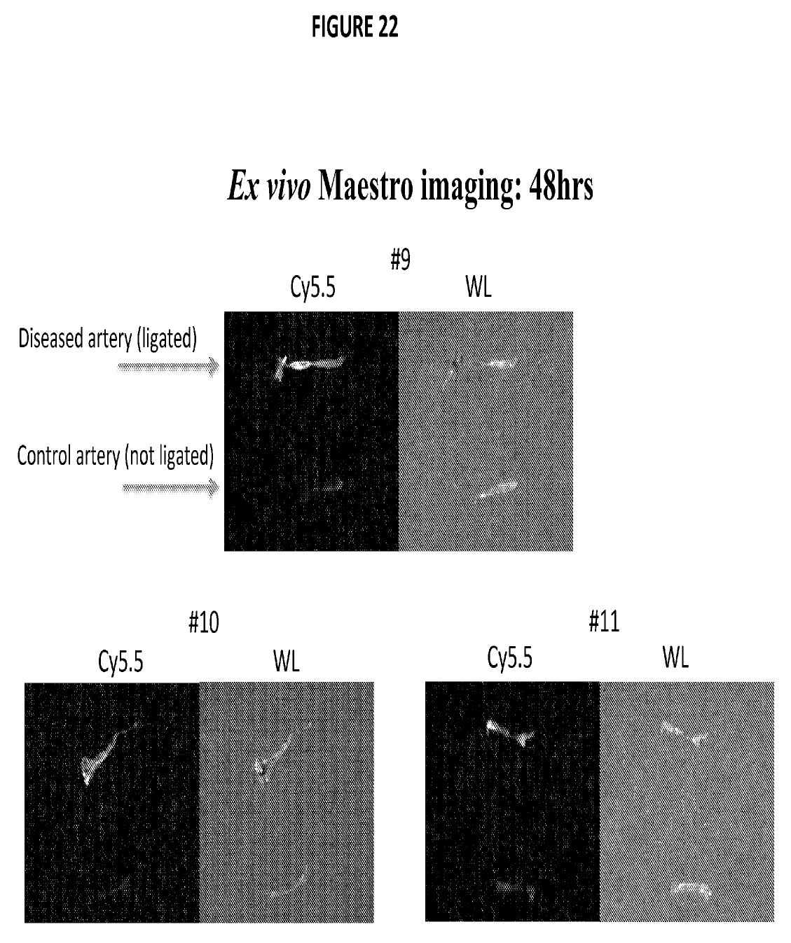

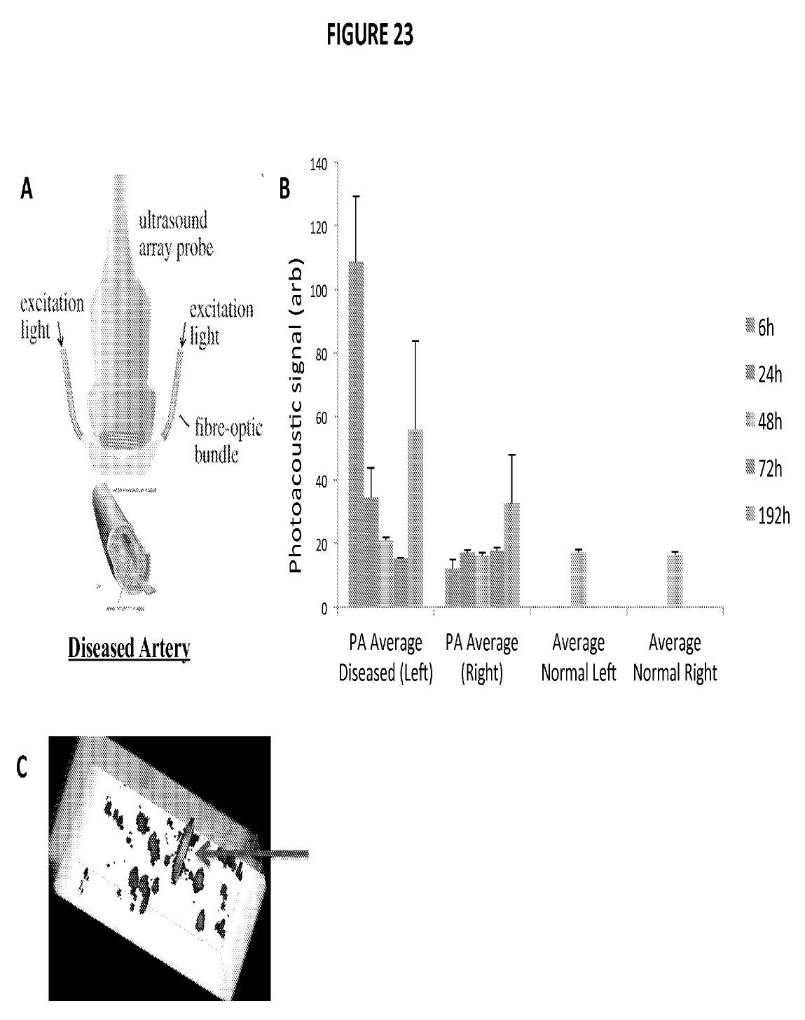

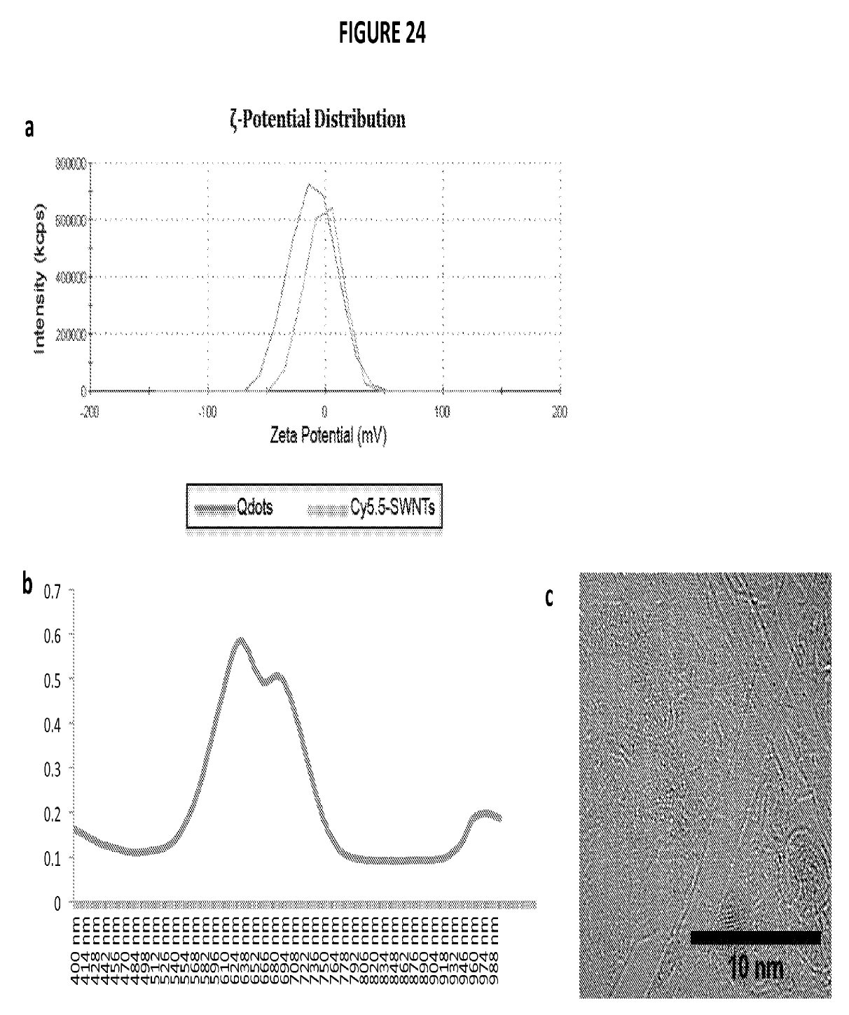

FIG. 21 shows a diseased, atherosclerotic carotid artery in comparison to a healthy nonsclerotic carotid artery in mice, where the diseased artery experiences a massive infiltration of macrophages that exclusively pick up SWNTs, in comparison to the healthy artery. FIG. 21, furthermore, illustrates that macrophages-Foam Cells and Ly-6C^{hi} monocytes are the only targets for SWNTs in atheromatous plaques. Corroborating the results described in FIG. 21, FIG. 22 illustrates that SWNTs migrate, most likely through Ly-6C^{hi} monocytes, more efficiently to the diseased artery than to the healthy artery.

In FIG. 23, photoacoustic imaging of diseased versus healthy arteries in mice is illustrated as an alternative detection methodology for SWNTs in vivo.

FIG. 24 illustrates SWNT distribution when conjugated to Cy5.5; 10-300 Cy5.5 dyes can be conjugated to a SWNT.

While fluorescent dyes as well as photoacoustic imaging can be used to view and follow SWNTs, other means are available for detecting SWNTs in vivo and hence, tracing the location and fate of foamy macrophages and Ly-6C^{hi} monocytes that have internalized SWNTs such as Raman Imaging and Magnetic Resonance Imaging, by imbibing Gadolinium [Gd] into the SWNTs, as described by Sitharaman et al., 2005. In conclusion, the large amount of foamy macrophages and Ly-6C^{hi} monocytes that pick up SWNTs and accumulate in atheromatous plaques enable exceptional methods for detecting tumorous as well as atherosclerotic tissues and for delivery of therapeutic drugs that are attached to the SWNTs to those tissues.

### REFERENCES

Adams J M (1985). The c-myc oncogene driven by immunoglobulin enhancers induces lymphoid malignancy in transgenic mice. Nature 318(6046):533-538.
Andriole G L et al. (2009). Mortality results from a randomized prostate-cancer screening trial. N Engl J Med 360:1310-1318.
Bertwistle D and Sherr C J (2007). Regulation of the Arf tumor suppressor in Eμ-Myc transgenic mice: longitudinal study of Myc-induced lymphomagenesis. Blood 109:792-794.
Endo M et al. (2008). Potential applications of carbon nanotubes. Topics in Applied Physics 111:13-61.
Gannon C J et al. (2007). Carbon nanotube-enhanced thermal destruction of cancer cells in a noninvasive radiofrequency field. Cancer 110(12):2654-2665.
Herzenberg L A et al. (2006). Interpreting flow cytometry data: a guide for the perplexed. Nat Immunol 7:681-685.
Kosuge et al. (2012). Near Infrared Imaging and Photothermal Ablation of Vascular Inflammation Using Single-Walled Carbon Nanotubes. J Am Heart Assoc 2012, 1:e002568.
Li F C et al. (2004). Dorsal skinfold titanium chamber for non-invasive imaging in nude mice using multiphoton and harmonic generation microscopy. Biophotonics Conference, pp. 185-186.
Lindner J R (2010). Molecular Imaging of Myocardial and Vascular Disorders With Ultrasound FREE. J Am Coll Cardiol Img 3(2):204-211.
Liu Z et al. (2009). Preparation of carbon nanotube bioconjugates for biomedical application. Nat Protoc 4(9):1372-1382.
Mori S et al. (2008). Utilization of pathway signatures to reveal distinct types of B lymphoma in the Eμ-myc Model and human diffuse large B-cell lymphoma. Cancer Res 68: 8525.
Nagaraj S et al. (2010). Anti-inflammatory Triterpenoid Blocks Immune Suppressive Function of MDSCs and Improves Immune Response in Cancer. Clin Cancer Res 16: 1812-1823.
Nefedova Y et al. (2007). Mechanism of all-trans retinoic acid effect on tumor-associated myeloid-derived suppressor cells. Cancer Res 67:11021-11028.
Robinson J T et al. (2010). High performance in vivo near-IR (>1 μm) imaging and photothermal cancer therapy with cancer therapy with carbon nanotubes. Nano Res 3(11):779-793.
Roederer M (2001). Spectral compensation for flow cytometry: visualization artifacts, limitations and caveats. Cytometry 45:194-205.
Schipper M L et al. (2008). A pilot toxicology study of single-walled carbon nanotubes in a small sample of mice. Nat Nanotechnol 3:216-221.
Sitharaman B et al. (2005). Superparamagnetic gadonanotubes are high-performance MRI contrast agents. Chem Commun 3915-3919.
Sottoriva A et al. (2013). Intratumor heterogeneity in human glioblastoma reflects cancer evolutionary dynamics. PNAS 110(10):4009-4014.
Swirski F K et al (2007). Ly-6C^{hi} monocytes dominate hypercholesterolemia-associated monocytosis and give rise to macrophages in atheromata. 117(1):195-205.
Tiemessen M M et al. (2007). CD4+CD25+Foxp3+ regulatory T cells induce alternative activation of human monocytes/macrophages. Proc Natl Acad Sci USA 104:19446-19451.
Welsher K et al. (2009). A route to brightly fluorescent carbon nanotubes for near-infrared imaging in mice. Nat Nanotechnol 4(11): 773-780.
Yona S et al. (2009). Monocytes: subsets, origins, fates and functions.
Current Opinion in Hematology 17:53-59.
de la Zerda A et al. (2010). Ultra-High sensitivity carbon nanotube agents for photoacoustic molecular imaging in living mice. Nano Lett 10:2168-2172.
Zhuang et al. (2009). Supramolecular Stacking of Doxorubicin on Carbon Nanotubes for in vivo Cancer Therapy. Agnew Chem Int Ed Engl, 48(41):7668-7672.

What is claimed is:

1. A method of treating one or more tumors in a mammalian subject, wherein the mammalian subject comprises Ly-6C^{hi} monocytes or CD14⁺ monocytes, and wherein the method comprises:
administering to the mammalian subject single walled carbon nanotubes (SWNTs) conjugated to one or more anti-tumor therapeutic agents;
allowing the monocytes to internalize the SWNTs, thereby producing SWNT-carrying monocytes;
allowing the one or more tumors to take up the SWNT-carrying monocytes.

2. The method according to claim 1, wherein the one or more anti-tumor therapeutic agents comprises an agent selected from the group consisting of: an agent that causes DNA damage, an agent that inhibits RNA or DNA synthesis, a cytoskeleton-disrupting agent, a topoisomerase inhibitor, a nucleotide analog or precursor analog, a peptide antibiotic, a vinca alkaloid or derivative thereof, and a monoclonal antibody.

3. The method according to claim 1, wherein the one or more anti-tumor therapeutic agents comprises an agent that causes DNA damage selected from the group consisting of: an alkylating agent, cyclophosphamide, mechlorethamine, uramustine, melphalan, chlorambucil, ifosfamide, bendamustine, carmustine, lomustine, streptozocin, busulfan, a platinum-based agent, cisplatin, carboplatin, nedaplatin, oxaliplatin, satraplatin, and triplatin tetranitrate.

4. The method according to claim 1, wherein the one or more anti-tumor therapeutic agents comprises an agent selected from the group consisting of: daunorubicin, doxorubicin, epirubicin, idarubicin, valrubicin, mitoxantrone, paclitaxel, docetaxel, an epothilone, patupilone, sagopilone, ixabepilone, irinotecan, topotecan, etoposide, teniposide, tafluposide, azacitidine, azathioprine, capecitabine, cytarabine, doxifluridine, fluorouracil, gemcitabine, hydroxyurea, mercaptopurine, methotrexate, tioguanine, bleomycin, actinomycin, vinblastine, vincristine, vindesine, and vinorelbine.

5. The method according to claim 1, wherein the one or more anti-tumor therapeutic agents comprises a monoclonal antibody.

6. The method according to claim 5, wherein the monoclonal antibody is selected from the group consisting of: cetuximab, panitumab, rituximab, bevacizumab, ipilimumab, ofatumumab, and ocrelizumab.

7. The method according to claim 1, wherein the SWNTs are functionalized with a targeting peptide.

8. The method according to claim 7, wherein the targeting peptide comprises the amino acid sequence RGD.

9. The method according to claim 1, wherein the SWNTs are conjugated to a detectable label.

10. The method according to claim 1, wherein the one or more tumors comprises glioblastoma.

11. The method according to claim 1, wherein the one or more tumors comprises a B-cell lymphoma.

12. The method according to claim 1, wherein the mammalian subject is a human subject comprising CD14⁺ monocytes.

13. A method for locating atherosclerotic tissue in a mammalian subject, wherein the mammalian subject comprises Ly-6C^{hi} monocytes or CD14⁺ monocytes, and wherein the method comprises:
administering to the mammalian subject single walled carbon nanotubes (SWNTs) conjugated to a detectable label;
allowing the monocytes to internalize the SWNTs, thereby producing SWNT-carrying monocytes;
allowing the atherosclerotic tissue to take up the SWNT-carrying monocytes; and
locating the SWNT-carrying monocytes taken up by the atherosclerotic tissue by detecting the detectable label.

14. The method according to claim 13, wherein the detectable label enables imaging of the SWNTs by a method selected from the group consisting of: fluorescence microscopy, Raman imaging, photoacoustic imaging, ultrasound imaging, near-infrared imaging, magnetic resonance imaging, radiolabel-based imaging, computed tomography, and X-ray imaging.

15. The method according to claim 14, wherein the detectable label is a photoacoustic imaging agent and the imaging is by photoacoustic imaging.

16. The method according to claim 13, wherein the SWNTs are further conjugated to one or more anti-atherosclerotic therapeutic agents.

17. The method according to claim 16, wherein the one or more anti-atherosclerotic therapeutic agents comprises an agent selected from the group consisting of: a statin, a fibrate, an inhibitors of the cyclooxygenase-2 pathway, an inhibitor of the arachidonate 5-lipoxygenase pathway, a bile acid sequestrant, niacin, probucol, a lysophosphatidic acid antagonist, and an acyl coenzyme A:cholesterol acyltransferase inhibitor.

18. The method according to claim 13, wherein the SWNTs are functionalized with a targeting peptide.

19. The method according to claim 18, wherein the targeting peptide comprises the amino acid sequence RGD.

20. The method according to claim 13, wherein the mammalian subject is a human subject comprising CD14⁺ monocytes.
